(19) United States
(12) Patent Application Publication
(10) Pub. No.: US 2007/0219237 A1
(43) Pub. Date: Sep. 20, 2007

(54) Chromane Derivatives

(75) Inventors: Yukari Matsumoto, Chita-gun (JP); Hirohisa Shimokawa, Chita-gun (JP); Tatsuya Yamagishi, Chita-gun (JP)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/687,781

(22) Filed: Mar. 19, 2007

Related U.S. Application Data
(60) Provisional application No. 60/804,872, filed on Jun. 15, 2006.
(60) Provisional application No. 60/783,663, filed on Mar. 17, 2006.

(51) Int. Cl. A61K 31/4745; C07D 471/02

(52) U.S. Cl. 514/303; 546/119

(57) ABSTRACT

This invention relates to compounds of the formula (I):

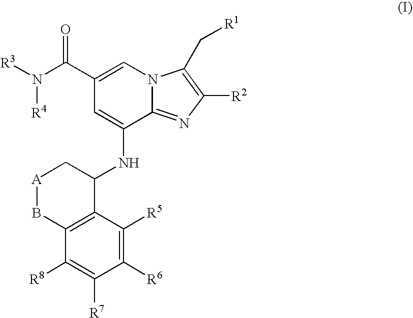

or a pharmaceutically acceptable salt thereof, wherein:
R¹, R², R³, R⁴, R⁵, R⁶, R⁷, R⁸ A and B are each as described herein or a pharmaceutically acceptable salt, and compositions containing such compounds and the method of treatment and the use, comprising such compounds for the treatment of a condition mediated by acid pump antagonistic activity such as, but not limited to, as gastrointestinal disease, gastroesophageal disease, gastroesophageal reflux disease (GERD), laryngopharyngeal reflux disease, peptic ulcer, gastric ulcer, duodenal ulcer, NSAID-induced ulcers, gastritis, infection of Helicobacter pylori, dyspepsia, functional dyspepsia, Zollinger-Ellison syndrome, non-erosive reflux disease (NERD), visceral pain, cancer, heartburn, nausea, esophagitis, dysphagia, hypersalivation, airway disorders or asthma.

### BACKGROUND OF THE INVENTION

This invention relates to chromane derivatives. These compounds have selective acid pump inhibitory activity. The present invention also relates to a pharmaceutical composition, method of treatment and use, comprising the above derivatives for the treatment of disease conditions mediated by acid pump modulating activity; in particular acid pump inhibitory activity.

It has been well established that proton pump inhibitors (PPIs) are prodrugs that undergo an acid-catalyzed chemical rearrangement that permits them to inhibit H⁺/K⁺-ATPase by covalently binding to its Cystein residues (Sachs, G. et. al., Digestive Diseases and Sciences, 1995, 40, 3S-23S; Sachs et. al., Annu Rev Pharmacol Toxicol, 1995, 35, 277-305.). However, unlike PPIs, acid pump antagonists inhibit acid secretion via reversible potassium-competitive inhibition of H⁺/K⁺-ATPase. SCH28080 is one of such reversible inhibitors and has been studied extensively. Other newer agents (revaprazan, soraprazan, AZD-0865 and CS-526) have entered in clinical trials confirming their efficacy in human (Pope, A.; Parsons, M., Trends in Pharmacological Sciences, 1993,14, 323-5; Vakil, N., Alimentary Pharmacology and Therapeutics, 2004, 19, 1041-1049.). In general, acid pump antagonists are found to be useful for the treatment of a variety of diseases, including gastrointestinal disease, gastroesophageal disease, gastroesophageal reflux disease (GERD), laryngopharyngeal reflux disease, peptic ulcer, gastric ulcer, duodenal ulcer, non-steroidal anti-inflammatory drug (NSAI D)-induced ulcers, gastritis, infection of Helicobacter pylori, dyspepsia, functional dyspepsia, Zollinger-Ellison syndrome, non-erosive reflux disease (NERD), visceral pain, cancer, heartburn, nausea, esophagitis, dysphagia, hypersalivation, airway disorders or asthma (hereinafter, referred as “APA Diseases”, Kiljander, Toni O, American Journal of Medicine, 2003, 115 (Suppl. 3A), 65S-71S; Ki-Baik Hahm et al., J. Clin. Biochem. Nutr., 2006, 38, (1), 1-8).

WO99155705, WO99/55706 and WO04/046144 disclose compounds reported to be acid pump antagonists. They refer to certain compounds having imidazo[1,2-a]pyridine structure.

There is a need to provide new acid pump antagonists that are good drug candidates and address unmet needs by PPIs for treating diseases. In particular, preferred compounds should bind potently to the acid pump whilst showing little affinity for other receptors and show functional activity as inhibitors of acid-secretion in stomach. They should be well absorbed from the gastrointestinal tract, be metabolically stable and possess favorable pharmacokinetic properties. They should be non-toxic. Furthermore, the ideal drug candidate will exist in a physical form that is stable, non-hygroscopic and easily formulated.

### SUMMARY OF THE INVENTION

In this invention, it has now been found out that the new class of compounds having a chromane moiety and imidazo[1,2-alpyridine structure substituted by (a hydroxy group or a moiety convertible into a hydroxy group in vivo)-methyl group on the 3-position showed acid pump inhibitory activity and favorable properties as drug candidates, and thus are useful for the treatment of disease conditions mediated by acid pump inhibitory activity such as APA Diseases.

The present invention provides a compound of the following formula (I):

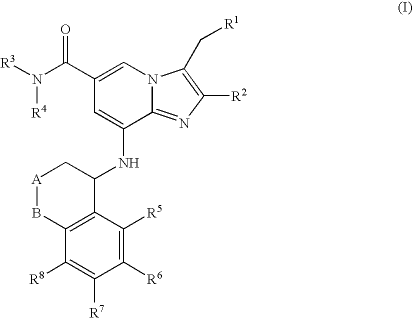

or a pharmaceutically acceptable salt thereof, wherein:
-A-B- represents —O—CH₂— or —CH₂—O—;
R¹ represents a hydroxy group or a moiety convertible into a hydroxy group in vivo;
R² represents a C₁-C₆ alkyl group;
R³ and R⁴ independently represent a C₃-C₆ alkyl group or a C₃-C₇ cycloalkyl group, said C₁-C₆ alkyl group and said C₃-C₇ cycloalkyl group being unsubstituted or substituted with 1 to 3 substituents independently selected from the group consisting of a halogen atom, a hydroxy group, a C₁-C₆ alkoxy group and a C₃-C₇ cycloalkyl group; or R³ and R⁴ taken together with the nitrogen atom to which they are attached form a 4 to 7 membered heterocyclic group being unsubstituted or substituted with 1 to 3 substituents selected from the group consisting of a hydroxy group, a C₁-C₆ alkyl group, a C₁-C₆ alkoxy group and a hydroxy-C₁-C₆ alkyl group; and
R⁵, R⁶, R⁷ and R⁸ independently represent a hydrogen atom, a halogen atom or a C₁-C₆ alkyl group.

Also, the present invention provides a pharmaceutical composition comprising a compound of formula (I) or a pharmaceutically acceptable salt thereof, each as described herein, together with a pharmaceutically acceptable carrier for said compound.

Also, the present invention provides a pharmaceutical composition comprising a compound of formula (I) or a pharmaceutically acceptable salt thereof, each as described herein, further comprising other pharmacologically active agent(s).

Also, the present invention provides a method for the treatment of a condition mediated by acid pump modulating activity in a mammalian subject including a human, which comprises administering to a mammal in need of such treatment a therapeutically effective amount of a compound of formula (I) or a pharmaceutically acceptable salt thereof, each as described herein.

Examples of conditions mediated by acid pump modulating activity include, but are not limited to, APA Diseases.

Further, the present invention provides the use of a compound of formula (I) or a pharmaceutically acceptable salt thereof each as described herein, for the manufacture of a medicament for the treatment of a condition mediated by acid pump inhibitory activity.

Further, the present invention provides a compound of formula (I) or a pharmaceutically acceptable salt thereof, for use in medicine.

Preferably, the present invention also provides the use of a compound of formula (I) or a pharmaceutically acceptable salt thereof, each as described herein, for the manufacture of a medicament for the treatment of diseases selected from APA Diseases.

The compounds of the present invention may show good acid pump inhibitory activity, less toxicity, good absorption, good distribution, good solubility, less protein binding affinity other than acid pump, less drug-drug interaction and good metabolic stability.

### DETAILED DESCRIPTION OF THE INVENTION

In the compounds of the present invention:

Where R², R³, R⁴, R⁵, R⁶, R⁷ and R⁸ are the C₁-C₆ alkyl group, this C₁-C₆ alkyl group may be a straight or branched chain group having one to six carbon atoms, and examples include, but are not limited to, methyl, ethyl, propyl, isopropyl, butyl, isobutyl, sec-butyl, tert-butyl, pentyl, 1-ethylpropyl and hexyl. Of these, C₁-C₂ alkyl is more preferred; methyl is more preferred.

Where R³ and R⁴ are the C₃-C₇ cycloalkyl group, this represents cycloalkyl group having three to seven carbon atoms, and examples include cyclopropyl, cyclobutyl, cyclopentyl, cyclohexyl and cycloheptyl. Of these, C₃-C₅ cycloalkyl group is preferred; cyclopropyl is more preferred.

Where the substituent of R³ and R⁴ are the C₁-C₆ alkoxy group, this represents the oxygen atom substituted with the said C₁-C₆ alkyl group, and examples include, but are not limited to, methoxy, ethoxy, propoxy, isopropoxy, butoxy, isobutoxy, sec-butoxy, tert-butoxy, pentyloxy and hexyloxy. Of these, a C₁-C₄ alkoxy is preferred; a C₁-C₂ alkoxy is preferred; methoxy is more preferred.

Where R³ and R⁴ taken together with the nitrogen atom to which they are attached form a 4 to 7 membered heterocyclic group, this 4 to 7 membered heterocyclic group represents a saturated heterocyclic group having three to six ring atoms selected from carbon atom, nitrogen atom, sulfur atom and oxygen atom other than said nitrogen atom, and examples include, but are not limited to, azetidinyl, pyrrolidinyl, imidazolidinyl, pyrazolidinyl, piperidyl, piperazinyl, hexahydroazepinyl, hexahydrodiazepinyl, morpholino, thiomorpholino and homomorpholino. Of these, azetidinyl, pyrrolidinyl, morpholino and homomorpholino are preferred; morpholino is more preferred.

Where the substituent of the 4 to 7 membered heterocyclic group is a hydroxy-C₁-C₆ alkyl group, this represents said C₁-C₆ alkyl group substituted with a hydroxy group, and examples include, but are not limited to, hydroxymethyl, 2-hydroxyethyl, 1-hydroxyethyl 3-hydroxypropyl, 2-hydroxypropyl, 2-hydroxy-1-methylethyl, 4-hydroxybutyl, 3-hydroxybutyl, 2-hydroxybutyl, 3-hydroxy-2-methylpropyl, 3-hydroxy-1-methylpropyl, 5-hydroxypentyl and 6-hydroxyhexyl. Of these, hydroxy-C₁-C₃ alkyl is preferred; hydroxymethyl is more preferred.

Where R⁵, R⁶, R⁷ and R⁸ are the halogen atom, it may be a fluorine, chlorine, bromine or iodine atom. Of these, a fluorine atom and a chlorine atom are preferred.

Where the “moiety convertible into a hydroxy group in vivo ” means a moiety transformable in vivo by e.g. hydrolysis and/or by an enzyme, e.g. an esterase, into a hydroxyl group. Examples of the moiety include, but are not limited to, ester and ether groups which may be hydrolyzed easily in vivo. Such moieties have known to those skilled in the art as pro-moieties' as described, for example, in “Design of Prodrugs” by H. Bundgaard (Elsevier, 1985). Preferred moieties convertible in vivo into a hydroxyl group are e.g. a C₁-C₆ alkoxy group, a C₁-C₆ alkyl-carbonyl-oxy group and a C₁-C₆ alkyl-carbonyl-oxy-methyl-oxy group.

Where -A-B— is —O—CH₂—, -A- corresponds —O— and —B— corresponds —CH₂—.

Where -A-B— is —CH₂—O—, -A- corresponds —CH₂— and —B— corresponds —O—.

The term “treating” and “treatment”, as used herein, refers to curative, palliative and prophylactic treatment, including reversing, alleviating, inhibiting the progress of, or preventing the disorder or condition to which such term applies, or one or more symptoms of such disorder or condition.

Preferred classes of compounds of the present invention are those compounds of formula (1) or a pharmaceutically acceptable salt thereof, each as described herein, in which:
(a) -A-B— is —O—CH₂— or —CH₂—O—:
(b) -A-B— is —CH₂—O—;
(c) R¹ is a hydroxy group, a C₁-C₆ alkoxy group or a C₁-C₆ alkyl-carbonyl-oxy group;
(d) R¹ is a hydroxy group;
(e) R² is a C₁-C₆ alkyl group;
(f) R² is a C₁-C₂ alkyl group;
(g) R² is a methyl group;
(h) R³ is a C₁-C₆ alkyl group;
(i) R³ is a C₁-C₂ alkyl group;
(j) R³ is a methyl group;
(k) R⁴ is a C₁-C₆ alkyl group being unsubstituted or substituted with a substituent selected from the group consisting of a hydroxy group and a C₁-C₆ alkoxy group;
(l) R⁴ is a C₁-C₂ alkyl group being unsubstituted or substituted with a substituent selected from the group consisting of a hydroxy group and a C₁-C₄ alkoxy group;
(m) R⁴ is a C₁-C₂ alkyl group being unsubstituted or substituted with a hydroxy group;
(n) R⁴ is a methyl group, an ethyl group or a 2-hydroxyethyl group:
(o) R³ and R⁴ taken together with the nitrogen atom to which they are attached form an azetidinyl group, a pyrrolidinyl group, a morpholino group or a homomorpholino group, said azetidinyl group, said pyrrolidinyl group, said morpholino group and said homomorpholino group being unsubstituted or substituted with 1 to 3 substituents selected from the group consisting of a hydroxy group, a C₁-C6 alkyl group, a C₁-C₆ alkoxy group and a hydroxy-C₁-C₆ alkyl group;
(p) R³ and R⁴ taken together with the nitrogen atom to which they are attached form a pyrrolidinyl group, a morpholino group or a homomorpholino group, said pyrrolidinyl group, said morpholino group and said homomorpholino group being unsubstituted or substituted with a substituent selected from the group consisting of a hydroxy group, a C₁-C₆ alkyl group, a C₁-C₆ alkoxy group and a hydroxy-C₁-C₆ alkyl group;
(q) R⁵, R⁶, R⁷ and R⁸ are independently a hydrogen atom, a halogen atom or a C₁-C6 alkyl group:
(r) R⁵, R⁶, R⁷ and R⁸ are independently a hydrogen atom, a halogen atom or a C₁-C₂ alkyl group;
(s) R⁵, R⁶, R⁷ and R⁸ are independently a hydrogen atom, a fluorine atom, a chlorine atom, or a methyl group;
(t) R⁵, R⁶, R⁷ and R⁸ are independently a hydrogen atom, a fluorine atom or a methyl group;
(u) R⁵ is a hydrogen atom, a fluorine atom or a methyl group;
(v) R⁶ is a hydrogen atom;
(w) R⁷ is a hydrogen atom or a fluorine atom; and
(x) R⁸ is a hydrogen atom;

Of these classes of compounds, any combination among (a) to (x) is also preferred.

Preferred compounds of the present invention are those compounds of formula (I) or a pharmaceutically acceptable salt thereof, each as described herein, in which:
(A) -A-B— is —O—CH₂— or —CH₂—O—; R¹ is a hydroxy group, C₁-C₆ alkoxy group or C₁-C₆ alkyl-carbonyl-oxy group; R² is a C₁-C₆ alkyl group; R³ and R⁴ are independently a C₁-C₆ alkyl group or a C₃-C₇ cycloalkyl group, said C₁-C₆ alkyl group and said C₁-C₇ cycloalkyl group being unsubstituted or substituted with 1 to 3 substituents independently selected from the group consisting of a halogen atom, a hydroxy group, a C₁-C₆ alkoxy group and a C₃-C₇ cycloalkyl group; or R³ and R⁴ taken together with the nitrogen atom to which they are attached form an azetidinyl group, a pyrrolidinyl group, a morpholino group or a homomorpholino group, said azetidinyl group, said pyrrolidinyl group, said morpholino group and said homomorpholino group being unsubstituted or substituted with 1 to 3 substituents selected from the group consisting of a hydroxy group, a C₁-C₆ alkyl group, a C₁-C₆ alkoxy group and a hydroxy-C₁-C₆ alkyl group; and R⁵, R⁶, R⁷ and R⁸ are independently a hydrogen atom, a halogen atom or a C₁-C6 alkyl group;
(B) -A-B— is —O—CH₂ or —CH₂—O—; R¹ is a hydroxy group; R², R³ and R⁴ are independently a C₁-C₆ alkyl group; or R³ and R⁴ taken together with the nitrogen atom to which they are attached form a morpholino group; R⁵ and R⁷ are independently a hydrogen atom, a halogen atom or a C₁-C₆ alkyl group; and R⁶ and R⁸ are independently a hydrogen atom or a halogen atom;
(C) -A-B— is —CH₂—O—; R¹ is a hydroxy group; R², R³ and R⁴ are independently a C₁-C₆ alkyl group; R⁵ and R⁷ are independently a hydrogen atom, a halogen atom or a C₁-C₆ alkyl group; and R⁶ and R⁸ are independently a hydrogen atom or a halogen atom; and
(D) -A-B— is —CH₂—O—; R¹ is a hydroxy group; R², R³ and R⁴ are each a methyl group; R⁵ and R⁷ are independently a hydrogen atom, a fluorine atom or a methyl group; and R⁶ and R⁸ are independently hydrogen atom or a fluorine atom.

The compounds of formula (I) containing one or more asymmetric carbon atoms can exist as two or more stereoisomers.

Included within the scope of the present invention are all stereoisomers and geometric isomers of the compounds of formula (I), including compounds exhibiting more than one type of isomerism, and mixtures of one or more thereof. Also included are acid addition salts wherein the counterion is optically active, for example, D-lactate or L-lysine, or racemate, DL-tartrate or DL-arginine.

One embodiment of the invention provides a compound selected from the group consisting of:
(S)-(−)-3-(Hydroxymethyl)-N,N,2-trimethyl-8-[(5-methyl-3,4-dihydro-2H-chromen-4-yl)amino]imidazo[1,2-a]pyridine-6-carboxamide;
(+)-8-(3,4-Dihydro-2H-chromen-4-ylamino)-3-(hydroxymethyl)-N,N,2-trimethylimidazo[1,2-a]pyridine-6-car boxamide;
(S)-(−)-8-[(5,7-Difluoro-3,4-dihydro-2H-chromen-4-yl)amino]-3-(hydroxymethyl)-N,N,2-trimethylimidazol[1,2-a]pyridine-6-carboxamide; and
(−)-8-[(5-Fluoro-3,4-dihydro-2H-chromen-4-yl)amino]-3-(hydroxymethyl)-N,N,2-trimethylimidazo [1,2-a]pyridine-6-carboxamide;
or a pharmaceutically acceptable salt thereof.

Pharmaceutically acceptable salts of a compound of formula (I) include the acid addition salts (including disalts) thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

For a review on suitable salts, see “Handbook of Pharmaceutical Salts: Properties, Selection, and Use” by Stahl and Wermuth (Wiley-VCH, Weinheim, Germany, 2002). A pharmaceutically acceptable salt of a compound of formula (I) may be readily prepared by mixing together solutions of the compound of formula (I) and the desired acid or base, as appropriate. The salt may precipitate from solution and be collected by filtration or may be recovered by evaporation of the solvent. The degree of ionization in the salt may vary from completely ionized to almost non-ionized.

Pharmaceutically acceptable salts of the compounds of the invention include both unsolvated and solvated forms. The term “solvate” is used herein to describe a molecular complex comprising a compound of the invention and one or more pharmaceutically acceptable solvent molecules, for example, ethanol. The term ‘hydrate’ is employed when said solvent is water.

Pharmaceutically acceptable solvates in accordance with the invention include hydrates and solvates wherein the solvent of crystallization may be isotopically substituted, e.g. D₂O, d₆-acetone, d₆-DMSO.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see J Pharm Sci, 64 (8), 1269-1288 by Haleblian (August 1975).

The compounds of formula (I) may exist in one or more crystalline forms. These polymorphs, including mixtures thereof are also included within the scope of the present invention.

The compounds of formula (I) containing one or more asymmetric carbon atoms can exist as two or more stereoisomers.

Included within the scope of the present invention are all stereoisomers of the compounds of formula (I), including compounds exhibiting more than one type of isomerism, and mixtures of one or more thereof.

The present invention includes all pharmaceutically acceptable isotopically-labeled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorin, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labeled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation a ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁵F, ¹⁵O and ¹³N, can be useful in Positron Emission Topography (PET) studies for examining substrate receptor occupancy.

Isotopicaliy-labeled compounds of formula (I) can generally be prepared by conventional techniques known to those skilled in the art or by processes analogous to those described in the accompanying examples and preparations using an appropriate isotopically-labeled reagents in place of the non-labeled reagent previously employed.

All of the compounds of the formula (I) can be prepared by the procedures described in the general methods presented below or by the specific methods described in the examples section and the preparations section, or by routine modifications thereof. The present invention also encompasses any one or more of these processes for preparing the compounds of formula (I), in addition to any novel intermediates used therein.

### General Synthesis

The compounds of the present invention may be prepared by a variety of processes well known for the preparation of compounds of this type, for example as shown in the following Method A.

Unless otherwise indicated, R¹, R², R³, R⁴, R⁵, R⁶, R⁷, R⁸, A and B in the following methods are as defined above. All starting materials in the following general syntheses may be commercially available or obtained by conventional methods known to those skilled in the art, such as WO99/55706 and WO 02/20523 and the disclosures of which are incorporated herein by references.

### Method A

This illustrates the preparation of compounds of formula (Ia) wherein R¹ is OH.

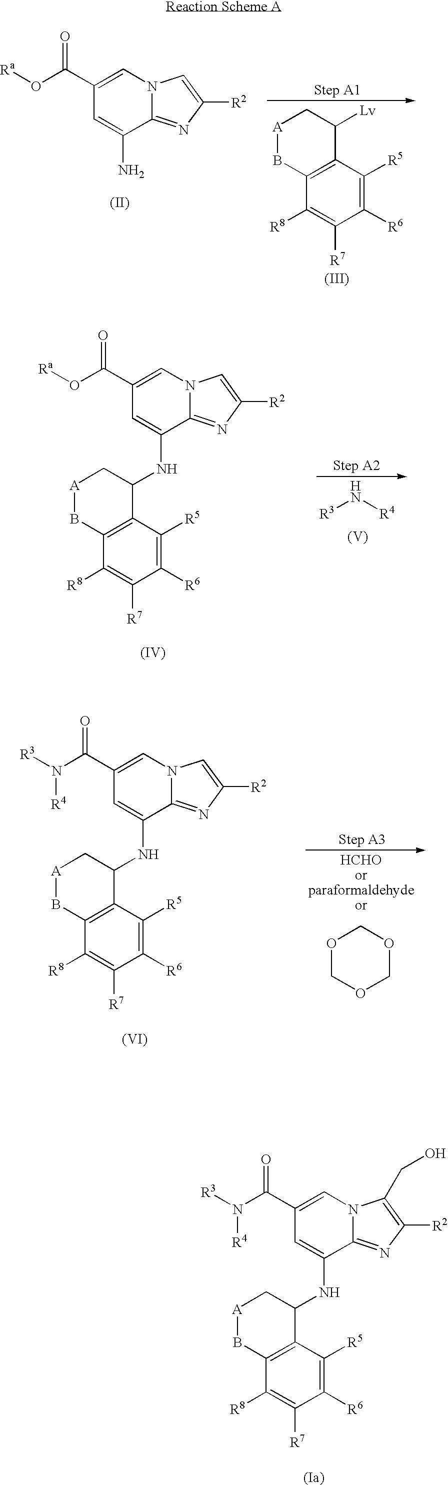

In Reaction Scheme A, R^{d} is a carboxy-protecting group; Lv is a leaving group; and the same shall apply hereinafter.

The term “leaving group”, as used herein, signifies a group capable of being substituted by nucleophilic groups, such as a hydroxy group, amines or carboanions and examples of such leaving groups include halogen atoms, an alkylsulfonyl group and a phenylsulfonyl group. Of these, a bromine atom, a chlorine atom, an iodine atom, a methylsulfonyl group, a trifluoromethylsulfonyl group and a 4-methylphenylsulfonyl group are preferred.

### Step A1

In this step, the compound of formula (IV) is prepared by nucleophilic substitution of the compound of formula (II), which is commercially available or may be prepared by the methods as described in WO99/55706 and WO02/020523 with the compound of formula (III), which is commercially available or may be prepared by the methods as described in WO2000/07851.

The reaction is normally and preferably effected in the presence of solvent. There is no particular restriction on the nature of the solvent to be employed, provided that it has no adverse effect on the reaction or the reagents involved and that it can dissolve reagents, at least to some extent. Examples of suitable solvents include: ethers, such as tetrahydrofuran (THF), ethylene glycol dimethyl ether and dioxane; amides, such as N,N-dimethylformamide (DMF), N,N-dimethylacetamide (DMA) and N-methyl-2-pyrrolidinone (NMP); nitrites, such as acetonitrile; ketones, such as acetone; alcohols, such as 2-methyl-2-propanol, 1-butanol, 1-propanol, 2-propanol, ethanol and methanol; and sulfoxide, such as dimethyl sulfoxide (DMSO). Of these solvents, amides, ketones and alcohols are preferred. Acetone is more preferred.

The reaction may be carried out with or without a base. There is likewise no particular restriction on the nature of the bases used, and any base commonly used in reactions of this type may equally be used here. Examples of such bases include: alkali metal alkoxides, such as sodium methoxide, sodium ethoxide and potassium tert-butoxide; alkali metal carbonates, such as lithium carbonate, sodium carbonate (Na₂CO₃), cesium carbonate and potassium carbonate (K₂CO₃); alkali metal hydrogencarbonates, such as sodium hydrogencarbonate (NaHCO₃) and potassium hydrogencarbonate; and organic amines, such as triethylamine, tripropylamine, tributylamine, dicyclohexylamine, N,N-diisopropylethylamine, N-methylpiperidine, N-methylmorpholine, 1,8-diazabicyclo[5.4.0]undec-7-ene (DBU) and 1,5-diazabicyclo{4.3.0]non-5-ene (DBN). Of these, potassium carbonate is preferred.

The reaction may be carried out with or without an iodide. Examples of such iodides include: sodium iodide, potassium iodide and cesium iodide. Of these, sodium iodide and potassium iodide are preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about 0° C. to about 250° C. The time required for the reaction may also vary widely, depending on many factors, notably the reaction temperature and the nature of the starting materials and solvent employed. However, provided that the reaction is effected under the preferred conditions outlined above, a period of from about 5 minutes to about 72 hours will usually suffice.

### Step A2

In this step, the compound of formula (VI) is prepared by (A2a1) hydrolysis of the compound of formula (IV) prepared as described in Step Al followed by (A2a2) condensing reaction with the compound of formula (V) or (A2b) substituting reaction of the compound of formula (IV) with the compound of formula (V).

### (A2a1) Hydrolysis

The reaction is normally and preferably effected in the presence of solvent. There is no particular restriction on the nature of the solvent to be employed, provided that it has no adverse effect on the reaction or the reagents involved and that it can dissolve reagents, at least to some extent. Examples of suitable solvents include: ether, such as tetrahydrofuran and dioxane; amides, such as N,N-dimethylformamide; alcohols, such as ethanol and methanol; and water; or mixed solvents thereof. Of these solvents, methanol, tetrahydrofuran and water are preferred.

The reaction is carried out in the presence of a base. There is likewise no particular restriction on the nature of the bases used, and any base commonly used in reactions of this type may equally be used here. Examples of such bases include: alkali metal hydroxides, such as lithium hydroxide (LiOH), sodium hydroxide (NaOH) and potassium hydroxide (KOH). Of these, sodium hydroxide is preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about 0° C. to about 100° C. The time required for the reaction may also vary widely, depending on many factors, notably the reaction temperature and the nature of the starting materials and solvent employed. However, provided that the reaction is effected under the preferred conditions outlined above, a period of from about 5 minutes to about 12 hours will usually suffice.

### (A2a2) Condensing Reaction

The reaction is normally and preferably effected in the presence of solvent. There is no particular restriction on the nature of the solvent to be employed, provided that it has no adverse effect on the reaction or the reagents involved and that it can dissolve reagents, at least to some extent. Examples of suitable solvents include: halogenated hydrocarbons, such as dichloromethane, chloroform, and 1,2-dichloroethane; ethers, such as tetrahydrofuran and dioxane; amides, such as N,N-dimethylformamide and N,N-dimethylacetamide; and nitrites, such as acetonitrile. Of these solvents, halogenated hydrocarbons and amides are preferred. Dichloromethane and N,N-dimethylformamide are more preferred.

The reaction is carried out in the presence of a condensing agent. There is likewise no particular restriction on the nature of the condensing agents used, and any condensing agents commonly used in reactions of this type may equally be used here. Examples of such condensing agents include: azodicarboxylic acid di-lower alkyl ester- triphenylphosphines, such as diethyl azodicarboxylate-triphenylphosphine; 2-halo-1-lower alkyl pyridinium halides, such as 2-chloro-1-methyl pyridinium iodide and 2-bromo-1-ethylpyridinium tetrafluoroborate (BEP); diarylphosphorylazides, such as diphenylphosphorylazide (DPPA); chloroformates, such as ethyl chloroformate and isobutyl chloroformate; phosphorocyanidates, such as diethyl phosphorocyanidate (DEPC); imidazole derivatives, such as N,N′-carbonyldiimidazole (CDI); carbodiimide derivatives, such as N,N-dicyclohexylcarbodiimide (DCC) and 1-(3-dimethylaminopropyl)-3-ethylcarbodiimide hydrochloride (EDCl); iminium salts, such as 2-(1H-benzotriazol-1-yl)-1,1,3,3-tetramethyluronium hexafluorophosphate (HBTU) and tetramethyl fluoroformamidinium hexafluoro phosphate (TFFH); and phosphonium salts, such as benzotriazol-1-yloxytris(dimethylamino)phosphonium hexafluorophosphate (BOP) and bromo-tris-pyrrolidino-phosphonium hexafluorophosphate (PyBrop). Of these, EDCl and HBTU are preferred.

Reagents, such as 4-(N,N-dimethylamino)pyridine (DMAP), and N-hydroxybenztriazole (HOBt), may be employed for this step. Of these, HOBt is preferred.

The reaction may be carried out with or without a base. There is likewise no particular restriction on the nature of the bases used, and any base commonly used in reactions of this type may equally be used here. Examples of such bases include: amines, such as N-methylmorpholine, triethylamine, diisopropylethylamine, N-methylpiperidine and pyridine. Of these, triethylamine and N-methylmorpholine are preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about 0° C. to about 80° C. The time required for the reaction may also vary widely, depending on many factors, notably the reaction temperature and the nature of the starting materials and solvent employed. However, provided that the reaction is effected under the preferred conditions outlined above, a period of from about 5 minutes to about 24 hours will usually suffice.

### (A2b) Substituting Reaction

The reaction can be carried out by heating the reactants in the neat amino compound or in an inert solvent under standard condition. There is no particular restriction on the nature of the solvent to be employed, provided that it has no adverse effect on the reaction or the reagents involved and that it can dissolve reagents, at least to some extent. Examples of suitable solvents include: ethers, such as ethylene glycol dimethyl ether, tetrahydrofuran and dioxane, amides, such as N,N-dimethylformamide and N,N-dimethylacetamide; nitriles, such as acetonitrile; and alcohols such as 2-methyl-2-propanol, 1-butanol, 1-propanol, 2-propanol, ethanol and methanol. Of these solvents, ethers and alcohols are preferred. Tetrahydrofuran is more preferred.

The reaction may be carried out with or without a catalyst. There is likewise no particular restriction on the nature of the catalysts used, and any catalysts commonly used in reactions of this type may equally be used here. Examples of such catalysts include: sodium cyanide or potassium cyanide. Of these, sodium cyanide is preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about 40° C. to about 200° C. The time required for the reaction may also vary widely, depending on many factors, notably the reaction temperature and the nature of the starting materials and solvent employed. However, provided that the reaction is effected under the preferred conditions outlined above, a period of from about 30 minutes to about 24 hours will usually suffice.

### Step A3

In this step, the desired compound of formula (Ia) is prepared by hydroxymethylation of the compound of formula (VI) prepared as described in Step A2 with formaldehyde, paraformaldehyde or 1,3,5-trioxane.

The reaction is carried out in the presence or absence of solvent. There is no particular restriction on the nature of the solvent to be employed, provided that it has no adverse effect on the reaction or the reagents involved and that it can dissolve reagents, at least to some extent. Examples of suitable solvents include: aliphatic hydrocarbons, such as hexane, heptane and petroleum ether; halogenated hydrocarbons, such as dichloromethane, chloroform, carbon tetrachloride and 1,2-dichloroethane; ethers, such as diethyl ether, diisopropyl ether, tetrahydrofuran and dioxane; aromatic hydrocarbons, such as benzene, toluene and nitrobenzene; amides, such as formamide, N,N-dimethylformamide, N,N-dimethylacetamide and hexamethylphosphoric triamide; amines, such as N-methylmorpholine, triethylamine, tripropylamine, tributylamine, diisopropylethylamine, dicyclohexylamine, N-methylpiperidine, pyridine, 4-pyrrolidinopyridine, N,N-dimethylaniline and N,N-diethylaniline; alcohols, such as methanol, ethanol, propanol, 2-propanol and 1-butanol; nitrites, such as acetonitrile and benzonitrile; sulfoxides, such as dimethyl sulfoxide and sulfolane; and water, Of these solvents, acetonitrile and water are preferred. The reaction is carried out in the presence of reagent, such as an acid or a base. There is likewise no particular restriction on the nature of the acids or bases used, and any acid or base commonly used in reactions of this type may equally be used here.

Examples of such acids include: carboxylic acids, such as acetic acid and propionic acid; inorganic acids, such as hydrochloric acid and sulfuric acid; organic acids, such as p-toluenesulfonic acid and trifluoro acetic acid; and Lewis acids, such as BF₃, AlCl₃, FeCl₃, AgCl, Znl₂, Fe(NO₃)₃, CF₃SO₃Si(CH₃)₃, Yb(CF₃ SO₃)₃ and SnCl₄. Of these, acetic acid is preferred.

Examples of such bases include: alkali metal acetates, such as lithium acetate, sodium acetate, potassium hydroxide and cesium acetate; alkali metal hydroxides, such as lithium hydroxide, sodium hydroxide and potassium hydroxide; alkali metal alkoxides, such as sodium methoxide, sodium ethoxide and potassium t-butoxide; alkali metal carbonates, such as lithium carbonate, sodium carbonate and potassium carbonate; alkali metal hydrogencarbonates, such as lithium hydrogencarbonate, sodium hydrogen carbonate and potassium hydrogencarbonate; and amines, such as N-methylmorpholine, triethylamine, tripropylamine, tributylamine, diisopropylethylamine, dicyclohexylamine, N-methylpiperidine, pyridine, 4-pyrrolidinopyridine, picoline, 4-(N,N-dimethylamino)pyridine, 2,6-di(t-butyl)-4-methylpyridine, quinoline, N,N-dimethylaniline, N,N-diethylaniline, DBN, 1,4-diazabicyclo[2.2.2]octane (DABCO), imidazole and DBU. Of these, sodium acetate is preferred

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about 0° C. to about 250° C. The time required for the reaction may also vary widely, depending on many factors, notably the reaction temperature and the nature of the starting materials and solvent employed. However, provided that the reaction is effected under the preferred conditions outlined above, a period of from about 5 minutes to about 72 hours will usually suffice.

The order of Step A2 and Step A3 can be replaced. For example, the compound whose 3 position is substituted with hydroxymethyl in the compound of the formula (IV) (wherein the compound is named compound (IVa) ) is prepared by hydroxymethylation of compound of the formula (IV) with formaldehyde, paraformaldehyde, or 1,3,5-trioxane as described in Step A3, and then, the compound of the formula (I) is prepared by reaction of the compound (IVa) with the compounds of formula (V) as described in Step A2.

### Method B

This illustrates the preparation of compounds of formula (Ia).

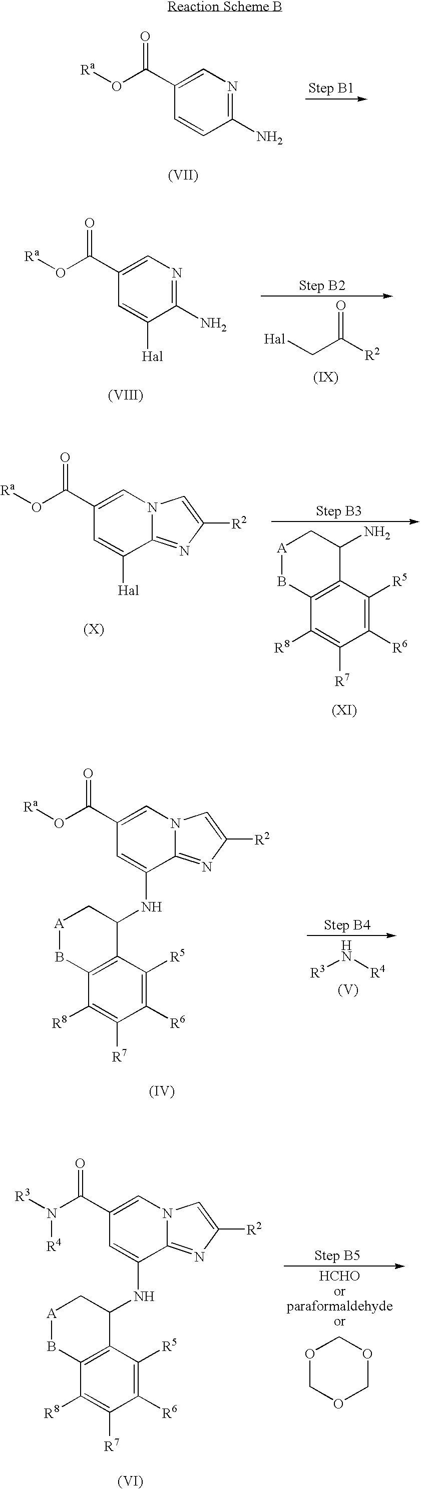

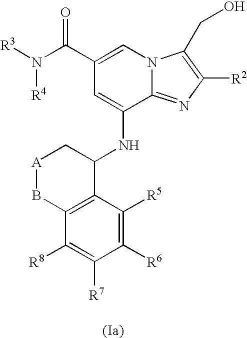

In Reaction Scheme B, Hal is a halogen atom; and the same shall apply hereinafter

### Step B1

In this step, the compound of formula (VIII) is prepared by halogenation of the compound of formula (VII), which is commercially available or may be prepared by the method as described in US2199839.

The reaction is normally and preferably effected in the presence of solvent. There is no particular restriction on the nature of the solvent to be employed, provided that it has no adverse effect on the reaction or the reagents involved and that it can dissolve reagents, at least to some extent. Examples of suitable solvents include: halogenated hydrocarbons, such as dichloromethane, chloroform, carbon tetrachloride and 1,2dichloroethane; ethers, such as diethyl ether, diisopropyl ether, tetrahydrofuran, cyclopentyl methyl ether and dioxane; aromatic hydrocarbons, such as benzene, toluene and nitrobenzene; amides, such as N,N-dimethylformamide, N,N-dimethylacetamide and hexamethylphosphoric triamide; nitriles, such as acetonitrile and benzonitrile; and carboxylic acid, such as acetic acid; or mixed solvents thereof. Of these, cyclopentyl methyl ether is preferred.

The reaction is carried out in the presence of a halogenating agent. There is likewise no particular restriction on the nature of the halogenating agents used, and any halogenating agent commonly used in reactions of this type may equally be used here. Examples of such halogenating agents include: chlorine, bromine, N-chlorosuccinimide, N-bromosuccinimide (NBS), tetra-n-butylammonium tribromide and 1,3-dibromo-5,5-dimethylhydantoin. Of these, N-bromosuccinimide is preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about 0° C. to about 80° C. The time required for the reaction may also vary widely, depending on many factors, notably the reaction temperature and the nature of the starting materials and solvent employed. However, provided that the reaction is effected under the preferred conditions outlined above, a period of from about 10 minutes to about 8 hours will usually suffice.

### Step B2

In this step, the compound of formula (X) is prepared by cyclization of the compound of formula (VIII) and the compound of formula (IX), which is commercially available.

The reaction is normally and preferably effected in the presence or absence of solvent. There is no particular restriction on the nature of the solvent to be employed, provided that it has no adverse effect on the reaction or the reagents involved and that it can dissolve reagents, at least to some extent. Examples of suitable solvents include: halogenated hydrocarbons, such as dichloromethane, chloroform, carbon tetrachloride and 1,2-dichloroethane; ethers, such as diethyl ether, diisopropyl ether, tetrahydrofuran and dioxane; aromatic hydrocarbons, such as benzene, toluene and nitrobenzene; amides, such as formamide, N,N-dimethylformamide, N,N-dimethylacetamide and hexamethylphosphoric triamide; ketones, such as acetone and 2-butanone; alcohols, such as methanol and ethanol; carboxylic acids, such as acetic acid; and nitrites, such as acetonitrile and propionitrile; or mixed solvents thereof. Of these, propionitrile is preferred.

The reaction may be carried out in the presence or absence of reagent, such as an acid or a base. There is likewise no particular restriction on the nature of the acids or bases used, and any acid or base commonly used in reactions of this type may equally be used here. Examples of such acids include: acids, such as hydrochloric acid, sulfuric acid, hydrobromic acid and p-toluenesulfonic acid. Of these, p-toluenesulfonic acid or the absence of acid is preferred. Examples of such bases include: alkali metal hydrogencarbonates, such as sodium hydrogencarbonate and potassium hydrogencarbonate; alkali metal carbonates, such as sodium carbonate and potassium carbonate; amines, such as triethylamine and diisopropylethylamine. Of these, diisopropylethylamine or the absence of base is preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about 20° C. to about 150° C. The time required for the reaction may also vary widely, depending on many factors, notably the reaction temperature and the nature of the starting materials and solvent employed. However, provided that the reaction is effected under the preferred conditions outlined above, a period of from about 3 hours to about 120 hours, will usually suffice.

### Step B3

In this step, the compound of formula (IV) is prepared by cross coupling of the compound of formula (X) with the compound of formula (XI), which may be commercially available or may be prepared by the methods described in the following Method C. The reaction is carried out under the same conditions as described in J. Am. Chem. Soc., 1996, 118, 7215.

The reaction is normally effected in the presence or absence of solvent. Typical solvent is aromatic hydrocarbons, such as benzene and toluene.

The reaction is carried out in the presence of a base. Typical base is sodium t-butoxide, as described in the literature indicated above.

The reaction is carried out in the presence of a catalyst. The catalyst consists of a palladium source, such as tris(dibenzylideneacetone)dipalladium (Pd₂(dba)₃), and a ligand, such as tri( otolyl)phosphine, 1,1′-binaphthalene-2,2′-diylbis(diphenylphosphine) (BINAP) and 1,1′-bis(diphenylphosphino)ferrocene (DPPF). Of these, combination of Pd₂(dba)₃ and BINAP is preferred according to the literature indicated above.

The reaction takes place typically in a range of 80° C. and 100° C. The time required for the reaction may vary widely, depending on the reaction temperature and the nature of the starting materials and catalyst employed. However, provided that the reaction is effected under the preferred conditions outlined above, a period of from about 1 hour to 22 hour will usually suffice.

### Step B4

In this step, the compound of formula (VI) is prepared by hydrolysis of the compound of formula (IV) prepared followed by condensing reaction with the compound of formula (V) or substituting reaction of the compound of formula (IV) with the compound of formula (V). The reaction may be carried out under the same condition as described in Step A2 of Method A.

### Step B5

In this step, the desired compound of formula (Ia) is prepared by hydroxymethylation of the compound of formula (VI) prepared as described in Step B2 with formaldehyde, paraformaldehyde or 1,3,5-trioxane. The reaction may be carried out under the same condition as described in Step A3 of Method A.

The order of Step B4 and Step B5 can be replaced. For example, the compound whose 3 position is substituted with hydroxymethyl in the compound of the formula (IV) (wherein the compound is named compound (IVa)) is prepared by hydroxymethylation of compound of the formula (IV) with formaldehyde, paraformaldehyde, or 1,3,5-trioxane as described in Step A3 of Method A, and then, the compound of the formula (la) is prepared by reaction of the compound (IVa) with the compounds of formula (V) as described in Step A2 of Method A.

The compound of formula (Ib) where R¹ is other than OH may be prepared by conventional methods known to those skilled in the art, written in such as “Design of Prodrugs” by H. Bundgaard (Elsevier, 1985).

### Method C

This illustrates the preparation of compounds of formula (XIa) wherein A is CH₂.

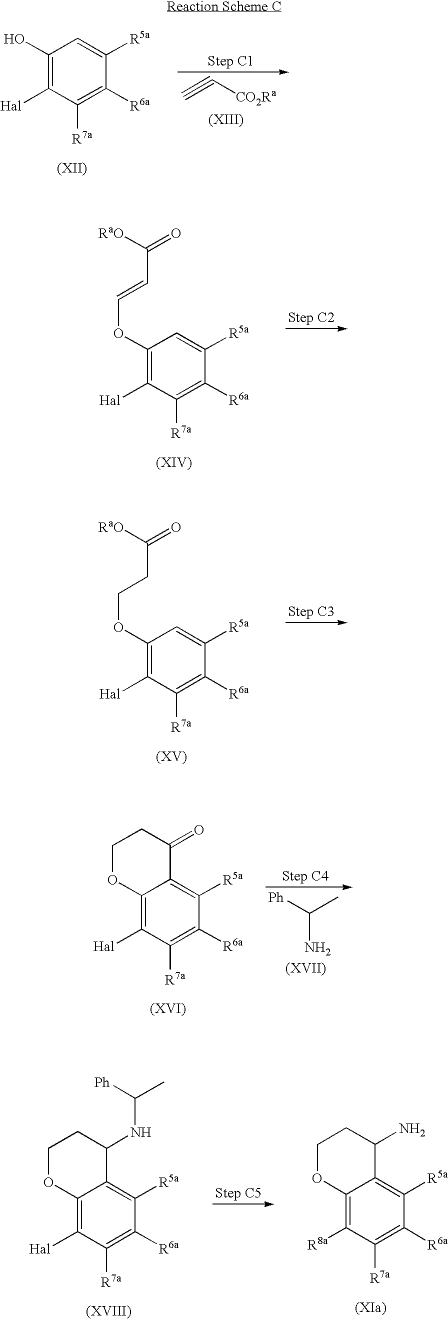

In Reaction Scheme C, R^{5a}, R^{6a} and R^{7a} are a hydrogen atom, a C₁-C₃ alkyl group or a fluorine atom; R^{8a} is a hydrogen atom or a fluorine atom.

### Step C1

In this step, the compound of formula (XIV) is prepared by addition reaction of the compound of formula (XII), which is commercially available, with the compound of formula (XIII), which is commercially available.

The reaction is normally and preferably effected in the presence of solvent There is no particular restriction on the nature of the solvent to be employed, provided that it has no adverse effect on the reaction or the reagents involved and that it can dissolve reagents, at least to some extent. Examples of suitable solvents include: halogenated hydrocarbons, such as dichloromethane, chloroform, carbon tetrachloride and 1,2-dichloroethane; ethers, such as diethyl ether, diisopropyl ether, tetrahydrofuran and dioxane; aromatic hydrocarbons, such as benzene, toluene and nitrobenzene; amides, such as formamide, N,N-dimethylformamide, N,N-dimethylacetamide and hexamethylphosphoric triamide; amines, such as N-methylmorpholine, triethylamine, tripropylamine, tributylamine, diisopropylethylamine, N-methylpiperidine, pyridine, 4-pyrrolidinopyridine, N,N-dimethylaniline and N,N-diethylaniline; alcohols, such as methanol, ethanol, propanol, 2-propanol and butanol; nitrites, such as acetonitrile and benzonitrile; sulfoxides, such as dimethyl sulfoxide and sulfolane; and ketones, such as acetone and diethylketone. Of these solvents, acetonitrile and tetrahydrofuran are preferred.

The reaction is carried out in the presence of a base. There is likewise no particular restriction on the nature of the bases used, and any base commonly used in reactions of this type may equally be used here. Examples of such bases include: alkali metal hydroxides, such as lithium hydroxide, sodium hydroxide and potassium hydroxide; alkali metal hydrides, such as lithium hydride, sodium hydride and potassium hydride; alkali metal alkoxides, such as sodium methoxide, sodium ethoxide and potassium t-butoxide; alkali metal carbonates, such as lithium carbonate, sodium carbonate and potassium carbonate; alkali metal hydrogencarbonates, such as lithium hydrogencarbonate, sodium hydrogencarbonate and potassium hydrogencarbonate; amines, such as N-methylmorpholine, triethylamine, tripropylamine, tributylamine, diisopropylethylamine, N-methylpiperidine, pyridine, 4-(N,N-dimethylamino)pyridine and DBU; and tetraalkylammonium fluorides, such as tetra-n-butylammonium fluoride (TBAF). Of these, TBAF is preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about 0° C. to about 100° C. The time required for the reaction may also vary widely, depending on many factors, notably the reaction temperature and the nature of the starting materials and solvent employed. However, provided that the reaction is effected under the preferred conditions outlined above, a period of from about 5 minutes to about 72 hours will usually suffice.

### Step C2

In this step, the compound of formula (XV) is prepared by hydrogenation of the compound of formula (XIV).

The reaction is normally and preferably effected in the presence of solvent. There is no particular restriction on the nature of the solvent to be employed, provided that it has no adverse effect on the reaction or the reagents involved and that it can dissolve reagents, at least to some extent. Examples of suitable solvents include: aromatic hydrocarbons, such as toluene; alcohols, such as methanol and ethanol; and carboxylic acids, such as acetic acid. Of these solvents, alcohols and carboxylic acids are preferred.

The reaction is carried out under hydrogen atmosphere and in the presence of a catalyst. There is likewise no particular restriction on the nature of the catalysts used, and any catalysts commonly used in reaction of this type may equally be used here. Examples of such catalysts include: palladium on carbon, platinum and Raney nickel. Of these catalysts, palladium on carbon is preferred.

In case that hydrodehalogenation (of substituent “Hal” in Reaction Scheme C) is a serious problem, the reaction may be carried out in the presence of an additive, which reduces activity of the catalyst employed. The additive is selected from substances known to show poisonous effect in some extent against the catalyst. Examples of such additives include: halide ion source, such as tetra-n-butylammonium bromide and sodium bromide; and sulfoxides, such as dimethylsulfoxide. Of these, sodium bromide is preferred.

The reaction can take place under a wide range of pressures, and precise pressure is not critical to the invention. The preferred pressure will depend upon such factors as the nature of the starting materials, and the solvent. However, in general, it is convenient to carry out the reaction at a pressure of from 1 atm to about 10 atm. The reaction can take place over a wide range of temperatures, and the precise reaction temperature is not critical to the invention. The preferred reaction temperature will depend upon such factors as the nature of the solvent and the starting materials. However, in general, it is convenient to carry out the reaction at a temperature of from about 0° C. to about 50° C. The time required for the reaction may also vary widely, depending on many factors, notably the pressure of hydrogen, the reaction temperature and the nature of the starting materials and solvent employed. However, provided that the reaction is effected under the preferred condition outlined above, a period of from about 30 minutes to about 12 hours will usually suffice.

### Step C3

In this step, the compound of formula (XVI) is prepared by cyclization of the compound of formula (XV).

The reaction is normally and preferably effected in the presence of an acid, which functions as solvent and reagent. There is no particular restriction on the nature of the acid to be employed, provided that it has no adverse effect on the reaction and that it can dissolve substrate, at least to some extent. Examples of suitable acids include: sulfuric acid and trifluoromethanesulfonic acid. Of these, trifluoromethanesulfonic acid is preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about 0° C. to about 150° C. The time required for the reaction may also vary widely, depending on many factors, notably the reaction temperature and the nature of the starting materials and solvent employed. However, provided that the reaction is effected under the preferred conditions outlined above, a period of from about 30 minutes to about 5 hours, will usually suffice.

### Step C4

In this step, the compound of formula (XVIII) is prepared by reductive amination of the compound of formula (XVI) with the compound of formula (XVII), which is commercially available. In case of using optically active compound of formula (XVII), the resulting compound of formula (XVIII) may be obtained as an optically active compound.

The reaction is normally and preferably effected in the presence of solvent. There is no particular restriction on the nature of the solvent to be employed, provided that it has no adverse effect on the reaction or the reagents involved and that it can dissolve reagents, at least to some extent. Examples of suitable solvents include: halogenated hydrocarbons, such as dichloromethane and 1,2-dichloroethane; ethers, such as diethyl ether, diisopropyl ether, tetrahydrofuran and dioxane; aromatic hydrocarbons, such as benzene and toluene; amides, such as formamide, N,N-dimethylformamide, N,N-dimethylacetamide and hexamethylphosphoric triamide; amines, such as N-methylmorpholine, triethylamine, tripropylamine, tributylamine, diisopropylethylamine, dicyclohexylamine, N-methylpiperidine, pyridine, 4-pyrrolidinopyridine, N,N-dimethylaniline and N,N-diethylaniline; and alcohols, such as methanol, ethanol, propanol, 2-propanol and butanol. Of these solvents, tetrahydrofuran is preferred.

The reaction is carried out in the presence or absence of a dehydrating agent. There is likewise no particular restriction on the nature of the dehydrating agents used, and any dehydrating agents commonly used in reactions of this type may equally be used here. Examples of such dehydrating agents include: titanium(IV) isopropoxide, magnesium sulfate and molecular sieves. Of these, titanium(IV) isopropoxide is preferred.

The reaction is carried out in the presence of a reducing agent. There is likewise no particular restriction on the nature of the reducing agents used, and any reducing agent commonly used in reactions of this type may equally be used here. Examples of such reducing agents include: metal borohydrides such as sodium borohydride and sodium cyanoborohydride; combinations of a hydrogen supplier, such as hydrogen gas and ammonium formate; catalysts, such as palladium-carbon, platinum and Raney nickel; a combination of metals, such as zinc and iron; acids, such as hydrochloric acid, acetic acid and acetic acid-ammonium chloride complex; hydride compounds such as lithium aluminum hydride, sodium borohydride and diisobutyl aluminum hydride; and borane reagents, such as boran-tetrahydrofuran complex, boran-dimethyl sulfide complex (BMS) and 9-borabicyclo[3,3,1]nonane (9-BBN). Of these, sodium borohydride is preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about −40° C. to about 20° C. The time required for the reaction may also vary widely, depending on many factors, notably the reaction temperature and the nature of the starting materials and solvent employed. However, provided that the reaction is effected under the preferred conditions outlined above, a period of from about 30 minutes to about 24 hours, will usually suffice.

### Step C5

In this step, the compound of formula (XIa) is prepared by hydrogenolysis of the compound of formula (XVIII).

The reaction is normally and preferably effected in the presence of solvent. There is no particular restriction on the nature of the solvent to be employed, provided that it has no adverse effect on the reaction or the catalyst involved and that it can dissolve reagents, at least to some extent. Examples of suitable solvents include: ethers, such as diethyl ether, diisopropyl ether, tetrahydrofuran and dioxane; aromatic hydrocarbons, such as benzene and toluene, alcohols, such as methanol, ethanol, propanol, 2-propanol and butanol; and carboxylic acids, such as acetic acid; or mixed solvents thereof. Of these, methanol is preferred.

The reaction is carried out in the presence of a hydrogen supplier and a catalyst. There is likewise no particular restriction on the nature of the hydrogen suppliers and the catalysts used, and any hydrogen suppliers and catalysts commonly used in reactions of this type may equally be used here. Examples of such hydrogen suppliers include hydrogen gas and ammonium formate. Of these, hydrogen gas is preferred. Examples of such catalysts include: palladium on carbon, palladium hydroxide and palladium chloride. Of these, palladium on carbon is preferred.

The reaction can take place under a wide range of pressures, and precise pressure is not critical to the invention. The preferred pressure will depend upon such factors as the nature of the starting materials, and the solvent. However, in general, it is convenient to carry out the reaction at a pressure of from 1 atm to about 10 atm. The reaction can take place over a wide range of temperatures, and the precise reaction temperature is not critical to the invention. The preferred reaction temperature will depend upon such factors as the nature of the solvent and the starting materials. However, in general, it is convenient to carry out the reaction at a temperature of from about 20° C. to about 100° C. The time required for the reaction may also vary widely, depending on many factors, notably the pressure of hydrogen, the reaction temperature and the nature of the starting materials and solvent employed. However, provided that the reaction is effected under the preferred condition outlined above, a period of from about 30 minutes to about 12 hours will usually suffice.

The preparation/isolation of individual enantiomers can be prepared by conventional techniques, such as chiral synthesis from a suitable optically pure precursor which may be prepared according to the Method C or resolution of the racemate (or the racemate of a salt or derivative) using, for example, chiral high-pressure liquid chromatography (HPLC).

Alternatively, a method of optical resolution of a racemate (or a racemic precursor) can be appropriately selected from conventional procedures, for example, preferential crystallization, or resolution of diastereomeric salts between a basic moiety of the compound of formula (I) and a suitable optically active acid such as tartaric acid.

The compounds of formula (I), and the intermediates in the above-mentioned preparation methods can be isolated and purified by conventional procedures, such as distillation, recrystallization or chromatographic purification.

Compounds of the invention intended for pharmaceutical use may be administered as crystalline or amorphous products. They may be obtained, for example, as solid plugs, powders, or films by methods such as precipitation, crystallization, freeze-drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a pharmaceutical composition or formulation in association with one or more pharmaceutically acceptable carriers or excipients. The term “carrier” or “excipient” is used herein to describe any ingredient other than the compound(s) of the invention. The choice of carrier or excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

### Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as, for example, tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include, for example, suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from about 1 wt % to about 80 wt % of the dosage form, more typically from about 5 wt % to about 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from about 1 wt % to about 25 wt %, preferably from about 5 wt % to about 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface-active agents, such as sodium lauryl sulfate and polysorbate 80, and glidants such as silicon dioxide and talc. When present, surface active agents may comprise from about 0.2 wt % to about 5 wt % of the tablet, and glidants may comprise from about 0.2 wt % to about 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from about 0.25 wt % to about 10 wt %, preferably from about 0.5 wt % to about 3 wt % of the tablet.

Other possible ingredients include anti-oxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tablefting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO00/35298.

### Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from about 3 to about 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

### Topical Administration

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated - see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

### Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurized container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as I-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from about 1 μg to about 20 mg of the compound of the invention per actuation and the actuation volume may vary from about 1 μg to about 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration. Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from about 1 to about 100 μg of the compound of formula (I). The overall daily dose will typically be in the range about 50 μg to about 20 mg which may be administered in a single dose or, more usually, as divided doses throughout the day.

### Rectal/Intravaginal Administration

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

### Other Technologies

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in. WO91/11172, WO94/02518 and WO98/55148.

### Kit-of-Parts

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another To assist compliance, the kit typically comprises directions for administration and may be provided with a so-called memory aid.

### Dosage

For administration to human patients, the total daily dose of the compounds of the invention is typically in the range of about 0.05 mg to about 500 mg depending, of course, on the mode of administration, preferred in the range of about 0.1 mg to about 400 mg and more preferred in the range of about 0.5 mg to about 300 mg. For example, oral administration may require a total daily dose of from about 1 mg to about 300 mg, while an intravenous dose may only require from about 0.5 mg to about 100 mg. The total daily dose may be administered in single or divided doses.

These dosages are based on an average human subject having a weight of about 65 kg to about 70 kg. The physician will readily be able to determine doses for subjects whose weight falls outside this range, such as infants and the elderly.

### Combinations

As discussed above, a compound of the invention exhibits acid pump inhibitory activity. An acid pump antagonist of the present invention may be usefully combined with another pharmacologically active compound, or with two or more other pharmacologically active compounds, particularly in the treatment of gastroesophageal reflux disease. For example, an acid pump antagonist, particularly a compound of the formula (I), or a pharmaceutically acceptable salt thereof, as defined above, may be administered simultaneously, sequentially or separately in combination with one or more agents selected from:
(i) histamine H₂ receptor antagonists, e.g. ranitidine, lafutidine, nizatidine, cimetidine, famotidine and roxatidine;
(ii) proton pump inhibitors, e.g. omeprazole, esomeprazole, pantoprazole, rabeprazole, tenatoprazole, ilaprazole and lansoprazole;
(iii) oral antacid mixtures, e.g. Maalox®, Aludrox® and Gaviscon®;
(iv) mucosal protective agents, e.g. polaprezinc, ecabet sodium, rebamipide, teprenone, cetraxate, sucralfate, chloropylline-copper and plaunotol;
(v) anti-gastric agents, e.g. Anti-gastrin vaccine, itriglumide and Z-360;
(vi) 5-HT₃ antagonists, e.g. dolasetron, palonosetron, alosetron, azasetron, ramosetron, mitrazapine, granisetron, tropisetron, E-3620, ondansetron and indisetron;
(vii) 5-HT₄ agonists, e.g. tegaserod, mosapride, cinitapride and oxtriptane;
(viii) laxatives, e.g. Trifyba®, Fybogel®, Konsyl®, Isogel®, Regulan®, Celevac® and Normacol®;
(ix) GABA_{B} agonists, e.g. baclofen and AZD-3355;
(x) GABA_{B} antagonists, e.g. GAS-360 and SGS-742;
(xi) calcium channel blockers, e.g. aranidipine, lacidipine, falodipine, azelnidipine, clinidipine, lomerizine, diltiazem, gallopamil, efonidipine, nisoldipine, amlodipine, lercanidipine, bevantolol, nicardipine, isradipine, benidipine, verapamil, nitrendipine, barnidipine, propafenone, manidipine, bepridil, nifedipine, nilvadipine, nimodipine and fasudil;
(xii) dopamine antagonists, e.g. metoclopramide, domperidone and levosulpiride;
(xiii) Tachykinin (NK) antagonists, particularly NK-3, NK-2 and NK-1 antagonists, e.g. nepadutant, saredutant, talnetant, (αR,9R)-7-[3,5-bis(trifluoromethyl)benzyl]-8,9,10,11-tetrahydro-9-methyl-5-(4-methylphenyl )-7H-[1,4]diazocino[2,1-g][1,7]naphthridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]methyl]-1,2-dihydro-3 H-1,2,4-triazol-3-one (MK-869), lanepitant, dapitant and 3-[[2-methoxy-5-(trifluoromethoxy)phenyl]methylamino]-2-phenyl-piperidine (2S, 3S);
(xiv) Helicobacter pylori infection agents, e.g. clarithromicyn, roxithromycin, rokitamycin, flurithromycin, telithromycin, amoxicillin, ampicillin, temocillin, bacampicillin, aspoxicillin, sultamicillin, piperacillin, lenampicillin, tetracycline, metronidazole, bithmuth citrate and bithmuth subsalicylate;
(xv) nitric oxide synthase inhibitors, e.g. GW-274150, tilarginine, P54, guanidioethyidisulfide and nitroflurbiprofen;
(xvi) vanilloid receptor 1 antagonists, e.g. AMG-517 and GW-705498;
(xvii) muscarinic receptor antagonists, e.g. trospium, solifenacin, tolterodine, tiotropium, cimetropium, oxitropium, ipratropium, tiquizium, dalifenacin and imidafenacin;
(xviii) calmodulin antagonists, e.g. squalamine and DY-9760;
(xix) potassium channel agonists, e.g. pinacidil, tillsolol, nicorandil, NS-8 and retigabine;
(xx) beta-1 agonists, e.g. dobutamine, denopamine, xamoterol, denopamine, docarpamine and xamoterol;
(xxi) beta-2 agonists, e.g. salbutamol; terbutaline, arformoterol, meluadrine, mabuterol, ritodrine, fenoterol, clenbuterol, formoterol, procaterol, tulobuterol, pirbuterol, bambuterol, tulobuterol, dopexamine and levosalbutamol;
(xxii) beta agonists, e.g. isoproterenol and terbutaline;
(xxiii) alpha 2 agonists, e.g. clonidine, medetomidine, lofexidine, moxonidine, tizanidine, guanfacine, guanabenz, talipexole and dexmedetomidine;
(xxiv) endthelin A antagonists, e.g. bonsetan, atrasentan, ambrisentan, clazosentan, sitaxsentan, fandosentan and darusentan;
(xxv) opioid p agonists, e.g. morphine, fentanyl and loperamide;
(xxvi) opioid p antagonists, e.g. naloxone, buprenorphine and alvimopan;
(xxvii) motilin agonists, e.g. erythromycin, mitemcinal, SLV-305 and atilmotin;
(xxviii) ghrelin agonists, e.g. capromorelin and TZP-101;
(xxix) AchE release stimulants, e.g. Z-338 and KW-5092;
(xxx) CCK-B antagonists, e.g. itrigiumide, YF-476 and S-0509;
(xxxi) glucagon antagonists, e.g. NN-2501 and A-770077;
(xxxii) piperacillin, lenampicillin, tetracycline, metronidazole, bithmuth citrate and bithmuth subsalicylate;
(xxxiii) Glucagon-like peptide-1 (GLP-1) antagonists, e.g. PNU-126814;
(xxxiv) small conductance calcium-activated potassium channel 3 (SK-3) antagonists, e.g. apamin, dequalinium, atracurium, pancuronium and tubocurarine;
(xxxv) mGluR5 anatagonists, e.g. ADX-10059 and AFQ-b 056;
(xxxvi) 5-HT3 agonists, e.g. pumosetrag(DDP733);
(xxxvii) mGluR8 agonists, e.g. (S)-3,4-DCPG and mGluR8-A.

### Method for Assessing Biological Activities:

The acid pump inhibitory activity and other biological activities of the compounds of this invention were determined by the following procedures. Symbols have their usual meanings: mL (milliliter(s)), μL (microlitter(s)), Kg (kirogram(s)), g (gram(s)), mg (milligram(s)), μg (microgram(s)), pmol (pico molar(s)), mmol (milli molar(s)), M (molar mass (m³/mol)), mM (milli molar mass), μM (micro molar mass), quant. (quantitative yield), nm (nanometer(s)), min (minute(s)), Cat# (catalog number), mV (millivolt(s)), ms (millisecond(s)), i.p. (intraperitoneal).

### Preparation of Gastric Vesicles from Fresh Porcine Stomachs

The porcine gastric vesicles for Porcine gastric H⁺/K⁺-ATPase inhibition assays were prepared from mucous membrane in fresh porcine stomachs by homogenization with a tight-fitted polytetrafluoroethylene (Teflone®) homogenizer in 0.25 M sucrose at 4° C. The crude pellet was removed with centrifugation at 20,000 g for 30 min. Then supernatant was centrifuged at 100,000 g for 30 min. The resulting pellet was re-suspended in 0.25 M sucrose, and then subjected to density gradient centrifugation at 132,000 g for 90 min. The gastric vesicles were collected from interface on 0.25 M sucrose layer containing 7% FicollTM PM400(Amersham Biosciences). This procedure was performed in a cold room.

### Ion-Leaky Porcine Gastric H⁺/K⁺-ATPase Inhibition

Ion-leaky porcine gastric H⁺/K⁺-ATPase inhibition was measured according to the modified method described in Biochemical Pharmacology, 1988, 37, 2231-2236.

The isolated vesicles were lyophilized, and then kept in deep-freezer until use. For enzyme assay, lyophilized vesicles were reconstituted with 3 mM MgSO₄ containing 40 mM Bis-tris (pH 6.4 at 37° C).

Enzyme reaction was performed incubating 5 mM KCl, 3 mM Na₂ATP, 3 mM MgSO₄ and 1.0 μg of reconstituted vesicles for 30 minutes at 37° C. in a final 60 μl of reaction mixture (40 mM Bis-tris, pH 6.4) with or without the test compound. Enzyme reaction was stopped by adding 10% sodium dodecyl sulphate (SDS). Released inorganic phosphate from ATP was detected by incubation with mixture of 1 part of 35 mM ammonium molybdate tetrahydrate in 15 mM Zinc acetate hydrate and 4 parts of 10% ascorbic acid (pH 5.0), resulting in phosphomolybdate, which has optical density at 750 nm. All example compounds showed potent inhibitory activity.

### In-Tight Porcine Gastric H⁺/K⁺-ATPase Inhibition

Ion-tight porcine gastric H⁺/K⁺-ATPase inhibition was measured according to the modified method described in Biochemical Pharmacology, 1988, 37, 2231-2236.

The isolated vesicles were kept in deep-freezer until use. For enzyme assay, vesicles were diluted with 3 mM MgSO₄ containing 5 mM Tris (pH 7.4 at 37° C.).

Enzyme reaction was performed incubating 150 mM KCl, 3 mM Na₂ATP, 3 mM MgSO₄, 15 μM valinomycin and 3.0 μg of vesicles for 30 minutes at 37° C. in a final 60 μl of reaction mixture (5mM Tris, pH 7.4) with or without the test compound. Enzyme reaction was stopped by adding 10% SDS. Released inorganic phosphate from ATP was detected by incubating with mixture of 1 part of 35 mM ammonium molybdate tetrahydrate in 15 mM Zinc acetate hydrate and 4 parts of 10% ascorbic acid (pH 5.0), resulting in phosphomolybdate, which has optical density at 750 nm.

The results of IC₅₀ values of the inhibitory activity for the compounds of following examples are shown in Table 1.

TABLE 1

| Example No. | IC₅₀(μM) |
|---|---|
| 1-1 | |
| 1-2 | 0.084 |
| 1-3 | 0.089 |
| 2-1 | 0.075 |
| 2-2 | 0.061 |
| 2-3 | 0.067 |
| 3-1 | |
| 3-2 | 0.037 |
| 3-3 | 0.041 |
| 4-2 | 0.029 |
| 4-3 | 0.030 |
| 6-2 | 0.043 |
| 6-3 | 0.061 |
| 7-1 | 0.140 |
| 7-2 | 0.096 |
| 7-3 | 0.110 |
| 8-1 | 0.055 |
| 8-2 | 0.040 |
| 8-3 | 0.052 |
| 9-1 | 0.047 |
| 9-2 | 0.072 |
| 9-3 | 0.061 |

All the tested compounds showed acid pump antagonistic activity.

### Canine Kidney Na⁺/K⁺-ATPase Inhibition

The powdered canine kidney Na⁺/K⁺-ATPase (Sigma) was reconstituted with 3 mM MgSO₄ containing 40 mM Tris (pH 7.4 at 37° C.). Enzyme reaction was performed incubating 100 mM NaCl, 2 mM KCl, 3 mM Na₂ATP, 3 mM MgSO₄ and 12 μg of enzyme for 30 minutes at 37° C. in a final 60 μl of reaction mixture (40 mM Tris, pH 7.4) with or without the test compound. Enzyme reaction was stopped by adding 10% SDS. Released inorganic phosphate from ATP was detected by incubating with mixture of I part of 35 mM ammonium molybdate tetrahydrate in 15 mM Zinc acetate hydrate and 4 parts of 10% ascorbic acid (pH 5.0), resulting in phosphomolybdate, which has optical density at 750 nm.

### Inhibition of Acid Secretion in the Gastric Lumen-Perfused Rat

Acid secretion in the gastric lumen-perfused rat was measured according to Watanabe et al. [Watanabe K et al., J. Physiol. (Paris) 2000; 94: 111-116]. Male Sprague-Dawley rats, 8 weeks old, deprived of food for 18 hours before the experiment with free access to water, were anesthetized with urethane (1.4 g/kg, i.p.) and tracheotomized. After a middle abdominal incision, a dual polyethylene cannula was inserted into the forestomach and the stomach was perfused with saline (37° C., pH 5.0) at a rate of 1 ml/min. The acid output in the perfusate was determined at 5 minutes interval by titration with 0.02 M NaOH to pH 5.0. After the determination of basal acid secretion for 30 min, the acid secretion was stimulated by a continuous intravenous infusion of pentagastrin (16 μg/kg/h). The test compounds were administered by an intravenous bolus injection or intraduodenal administration after the stimulated acid secretion reached a plateau phase. The acid secretion was monitored after the administration.

The activity was evaluated either inhibition of total acid secretion from 0 hours to 1.5 or 3.5 hours after administration or the maximum inhibition after administration.

The compound of Examples 1-9 showed a good inhibitory activity.

### Inhibition of Gastric Acid Secretion in the Heidenhain Pouch Dog

Male Beagle dogs weighing 7-15 kg with Heidenhain pouch [Heidenhain R: Arch Ges Physiol. 1879; 19: 148-167] were used. The animals were allowed to recover from surgery for at least three weeks before the experiments. The animals were kept at a 12 hour light-dark rhythm, housed singly. They received standard food once daily at 11:00 a.m. and tap water ad libitum, and were fasted overnight prior to the experiment, with free access to water. Gastric juice samples were collected throughout the experiment by gravity drainage every 15 min. Acidity in the gastric juice was measured by titration to the end point of pH 7.0. Acid secretion was stimulated by a continuous intravenous infusion of histamine (80 μg/kg/h). Oral or intravenous bolus administration of the test compounds was done 90 minutes after commencement of the histamine infusion. The acid secretion was monitored after the administration. The activity was evaluated by the maximum inhibition relative to the corresponding control value.

### Human Dofetilide Binding

Human ether a-go-go related gene (HERG) transfected HEK293S cells were prepared and grown in-house. Cell paste of HEK-293 cells expressing the HERG product can be suspended in 10-fold volume of 50 mM Tris buffer adjusted at pH 7.5 at 25° C. with 2 M HCl containing 1 mM MgCl₂, 10 mM KCl. The cells were homogenized using a Polytron homogenizer (at the maximum power for 20 seconds) and centrifuged at 48,000 g for 20 minutes at 4° C. The pellet was resuspended, homogenized and centrifuged once more in the same manner. The resultant supernatant was discarded and the final pellet was resuspended (10-fold volume of 50 mM Tris buffer) and homogenized at the maximum power for 20 seconds. The membrane homogenate was aliquoted and stored at -80° C. until use. An aliquot was used for protein concentration determination using a Protein Assay Rapid Kit (wako) and Spectra max plate reader (Wallac). All the manipulation, stock solution and equipment were kept on ice at all times. For saturation assays, experiments were conducted in a total volume of 200 μl. Saturation was determined by incubating 36 μl of [³H]-dofetilide, and 160 μl of membrane homogenates (20-30 μg protein per well) for 60 minutes at room temperature in the absence or presence of 10 μM dofetilide at final concentrations (4 μl) for total or nonspecific binding, respectively. All incubations were terminated by rapid vacuum filtration over PEI soaked glass fiber filter papers using Skatron cell harvester followed by two washes with 50 mM Tris buffer (pH 7.4 at 25° C.). Receptor-bound radioactivity was quantified by liquid scintillation counting using Packard LS counter.

For the competition assay, compounds were diluted in 96 well polypropylene plates as 4-point dilutions in semi-log format. All dilutions were performed in DMSO first and then transferred into 50 mM Tris buffer (pH 7.4 at 25° C.) containing 1 mM MgCl₂, 10 mM KCl so that the final DMSO concentration became equal to 1%. Compounds were dispensed in triplicate in assay plates (4 μl). Total binding and nonspecific binding wells were set up in 6 wells as vehicle and 10 μM dofetilide at final concentration, respectively The radioligand was prepared at 5.6× final concentration and this solution was added to each well (36 μl). The assay was initiated by addition of YSi poly-L-lysine SPA beads (50 μl, 1 mg/well) and membranes (110 μl, 20 μg/well). Incubation was continued for 60 minutes at room temperature. Plates were incubated for a further 3 hours at room temperature for beads to settle. Receptor-bound radioactivity was quantified by counting Wallac MicroBeta plate counter.

### Caco-2 Permeability

Caco-2 permeability was measured according to the method described in Shiyin Yee, Pharmaceutical Research, 763 (1997).

Caco-2 cells were grown on filter supports (Falcon HTS multiwell insert system) for 14 days. Culture medium was removed from both the apical and basolateral compartments and the monolayers were preincubated with pre-warmed 0.3 ml apical buffer and 1.0 ml basolateral buffer for 0.5 hour at 37° C. in a shaker water bath at 50 cycles/min. The apical buffer consisted of Hanks Balanced Salt Solution, 25 mM D-glucose monohydrate, 20 mM 2-morpholinoethanesulphonic acid (MES) Biological Buffer, 1.25 mM CaCl₂ and 0.5 mM MgCl₂ (pH 6.5). The basolateral buffer consisted of Hanks Balanced Salt Solution, 25 mM D-glucose monohydrate, 20 mM 2-[4-(2-hydroxyethyl)-1-piperazinyl]ethanesulfonic acid (HEPES) Biological Buffer, 1.25 mM CaCl₂ and 0.5 mM MgCl₂ (pH 7.4). At the end of the preincubation, the media was removed and test compound solution (10 μM) in buffer was added to the apical compartment. The inserts were moved to wells containing fresh basolateral buffer at 1 hour. Drug concentration in the buffer was measured by LC/MS analysis.

Flux rate (F, mass/time) was calculated from the slope of cumulative appearance of substrate on the receiver side and apparent permeability coefficient (Pₐₚₚ) was calculated from the following equation.

Pₐₚₚ (cm/sec)=(F×VD)/(SA×MD)

Where SA is surface area for transport (0.3 cm²), VD is the donor volume (0.3 ml), MD is the total amount of drug on the donor side at t=0. All data represent the mean of 2 inserts. Monolayer integrity was determined by Lucifer Yellow transport.

### Half-Life in Human Liver Microsomes (HLM)

Test compounds (1 μM) were incubated with 3.3 mM MgCl₂ and 0.78 mg/mL HLM (HL101) in 100 mM potassium phosphate buffer (pH 7.4) at 37° C. on the 96-deep well plate. The reaction mixture was split into two groups, a non-P450 and a P450 group. NADPH was only added to the reaction mixture of the P450 group. An aliquot of samples of P450 group was collected at 0, 10, 30, and 60 minutes time point, where 0 minutes time point indicated the time when NADPH was added into the reaction mixture of P450 group. An aliquot of samples of non-P450 group was collected at -10 and 65 minutes time point. Collected aliquots were extracted with acetonitrile solution containing an internal standard. The precipitated protein was spun down in centrifuge (2000 rpm, 15 min). The compound concentration in supernatant was measured by LC/MS/MS system.

The half-life value was obtained by plotting the natural logarithm of the peak area ratio of compounds/ internal standard versus time. The slope of the line of best fit through the points yields the rate of metabolism (k). This was converted to a half-life value using following equations:

Half-life=In 2/k

hERG Patch Clamp Assay

To determine the potential of compounds to inhibit the hERG channel, the cloned counterpart of the rapidly inactivating delayed rectifier potassium current (IKr).

HEK293 cells stably expressing the hERG channel were used in whole-cell patch clamp electrophysiology studies at ambient temperature (26.5-28.5° C.). The methodology for stable transfection of this channel in HEK293 cells can be found elsewhere (Zhou et al 1998, Biophysical Journal, 74, pp230-241). The solutions used for experimentation were standard extracellular solution of the following composition (mM); NaCl, 137; KCl, 4; CaCl₂, 1.8; MgCl₂, 1; Glucose, 10; HEPES, 10; pH 7.4±0.05 with NaOH/HCl; and standard intracellular solution of the following composition (mM); KCl, 130; MgCl₂, 1; HEPES, 10; EGTA, 5; MgATP, 5; pH 7.2±0.05 with KOH. The voltage protocol applied was designed to activate the hERG channel and allow the measurement of drug block of the channel and is as follows. First the membrane potential was stepped from a holding potential of −80 mV to +30 mV for 1s. This was followed by a descending voltage ramp at a rate of 0.5 mV/ms back to holding potential of −80 mV and the peak outward current observed during the repolarizing ramp was measured. This protocol was evoked repeatedly every 4 seconds (0.25 Hz). After establishing a stable baseline period in the presence of vehicle (0.1% v/v DMSO), four increasing concentrations of test compound were then bath-applied sequentially until the response reached steady-state or 10 minutes (whichever occurred first). 10 micromol/L dofetilide was used at the end of each experiment as an internal positive control and to define maximum block.

### Bioavailability in Rat

Adult rats of the Sprague-Dawley strain were used. One to two days prior to the experiments all rats were prepared by cannulation of the right jugular vein under anesthesia. The cannula was exteriorized at the nape of the neck. Blood samples (0.2-0.3 mL) were drawn from the jugular vein at intervals up to 24 hours after intravenous or oral administrations of the test compound. The samples were frozen until analysis. Bloavailability was assessed by calculating the quotient between the area under plasma concentration curve (AUC) following oral administration or intravenous administration.

### Bioavailability in Dog

Adult Beagle dogs were used. Blood samples (0.2-0.5 mL) were drawn from the cephalic vein at intervals up to 24 hours after intravenous or oral administrations of the test compound. The samples were frozen until analysis. Bioavailability was assessed by calculating the quotient between the area under plasma concentration curve (AUC) following oral administration or intravenous administration.

### Plasma Protein Binding

Plasma protein binding of the test compound (1 μM) was measured by the method of equilibrium dialysis using 96-well plate type equipment. Spectra-Por®, regenerated cellulose membranes (molecular weight cut-off 12,000-14,000, 22 mm×120 mm) were soaked for over night in distilled water, then for 20 minutes in 30% ethanol, and finally for 15 minutes in dialysis buffer (Dulbecco's phosphate buffered saline, pH7.4). Frozen plasma of human, Sprague-Dawley rats, and Beagle dogs were used. The dialysis equipment was assembled and added 150 μL of compound-fortified plasma to one side of each well and 150 μL of dialysis buffer to the other side of each well. After 4 hours incubation at 37° C. for 150 r.p.m, aliquots of plasma and buffer were sampled. The compound in plasma and buffer were extracted with 300 μL of acetonitrile containing internal standard compounds for analysis. The concentration of the compound was determined with LC/MSIMS analysis.

The fraction of the compound unbound was calculated by the following equation:

fu=1-{([plasma]_{eq}−[buffer]_{eq})/([plasma]_{eq})}

wherein [plasma]_{eq} and [buffer]_{eq} are the concentrations of the compound in plasma and buffer, respectively.

### Aqueous Solubility

Aqueous solubility in the mediums (a)-(c) was determined by following method:

Whatman mini-UniPrep chambers (Clifton, N.J., USA) containing more than 0.5 mg of compound and 0.5 mL of each medium were shaken overnight (over 8 hours) at room temperature. All samples were filtered through a 0.45 μm Polyvinylidene Difluoride (PVDF) membrane into the Whatman mini-UniPrep plunger before analysis. The filtrates were assayed by HPLC. <medium>(a) Simulated gastric fluid with no enzyme (SGN) at pH 1.2: Dissolve 2.0 g of NaCl in 7.0 mL of 10 M HCl and sufficient water to make 1000 mL; (b) Phosphate buffer saline (PBS) at pH 6.5: Dissolve 6.35 g of KH₂PO₄, 2.84 g of Na₂HPO₄ and 5.50 g of NaCl in sufficient water to make 1000 mL, the pH to 6.5: (c) 3.94 mg of sodium taurocholate (NaTC) and 1.06 mg of 1-palmitoyl-2-oleyl-L-phosphatidylcholine (POPC) in I mL of PBS (pH 6.5).

### Estimation of Hepatic Clearance Using the Metabolic Stability in Human Hepatocytes

Tested compounds (1 μM) were incubated statically with hepatocytes from human at 37° C. in a 95% air/5% CO₂ with target cell density of 0.5×10⁶ cells/ml and a total volume of 50 μL. Incubation was stopped at each time point by the addition of ice-cold acetonitrile (ACN). Aliquots of samples were mixed with 10% ACN containing an internal standard for LC/MS/MS analysis. After samples were sonicated for 10 minutes, samples were centrifuged at 2,000 rpm for 15 minutes, and then the supernatant was transferred to the other plates for analysis. The compound concentrations in supernatant were measured by LC/MS/MS system.

The disappearance rates of tested compounds were obtained by plotting the common logarithm of the peak area ratio of compounds I internal standard versus time. The slope of the line of best fit through the points yielded the rate of metabolism (ke). This value was scaled to take hepatocellularity, liver and body weight into account to give an intrinsic clearance value (CLᵢₙₜ) in ml/min/kg as illustrated in Equation 1. Hepatic clearance (CLₕ) was predicted from this intrinsic clearance value using the parallel tube model as shown in Equation 2. The predicted clearance divided by the hepatic blood flow (Qₕ) afforded the extraction ratio (Eₕ) (Equation 3).

kₑ×(g liver/kg body weight)×(ml incubations number of cells in incubation)×(cells/g liver) Equation 1

CLₕ=Qₕ×{1−exp (−CLᵢₙₜ/Qₕ)} Equation 2

Eₕ=CLₕ/Qₕ Equation 3

### Wherein, “gliver weight/kg body weight” is 21, “Cells/g liver” is 1.2×10⁸, “ml incubations/number of cells in incubation” is 2.0×10⁶, and Qₕ is 20 ml/min/kg.

Supposing that hepatic metabolism is the main route of drug elimination, systemic exposure (AUCₚₒ) after oral administration is calculated using Equation 4.

AUCₚₒ=Dose×(1−Eₕ)/CLₕ Equation 4

### EXAMPLES

The following examples are provided for the purpose of further illustration only and are not intended to be limitations on the disclosed invention. Unless stated on otherwise in the following examples, general experimental conditions are as follows: all operations were carried out at room or ambient temperature, that is, in the range of 18-25° C.; evaporation of solvent was carried out using a rotary evaporator under reduced pressure with a bath temperature of up to 60° C.; reactions were monitored by thin layer chromatography (TLC) and reaction times are given for illustration only; melting points (mp) given are uncorrected (polymorphism may result in different melting points); the structure and purity of all isolated compounds were assured by at least one of the following techniques: TLC (Merck silica gel 60 F₂₅₄ precoated TLC plates or Merck NH₂ gel (an amine coated silica gel) F₂₅₄ₛ precoated TLC plates), mass spectrometry, nuclear magnetic resonance spectra (NMR), infrared absorption spectra (IR) or microanalysis Yields are given for illustrative purposes only. Flash column chromatography was carried out using Biotage KP-SIL (40-63 μm), Biotage KP-NH (an amine coated silica gel) (40-75 μM) or Wako silica gel 300HG (40-60 μM). Preparative TLC was carried out using Merck silica gel 60 F₂₅₄ precoated TLC plates (0.5 or 1.0 mm thickness). All Mass data was obtained in Low-resolution mass spectral data (ESI) using ZMD™ or ZQ™ (Waters) and mass spectrometer NMR data were determined at 270 MHz (JEOL JNM-LA 270 spectrometer) or 300 MHz (JEOL JNM-LA300 spectrometer) using deuterated chloroform (99.8%) or dimethylsulfoxide (99.9%) as solvent unless indicated otherwise, relative to tetramethylsilane (TMS) as internal standard in parts per million (ppm); conventional abbreviations used are: s=singlet, d=doublet, m=multiplet, dd=doublet of doublet, sep=septet, br.s=broad singlet, br.d=broad doublet, etc. IR spectra were measured by a Fourier transform infrared spectrophotometer (Shimazu FTIR-8300). Optical rotations were measured using a P-1020 Digital Polarimeter (Japan Spectroscopic CO, Ltd.). The powder X-ray diffraction (PXRD) pattern was determined using a Rigaku RINT-TTR powder X-ray diffractometer fitted with an automatic sample changer, a 2 theta-theta goniometer, beam divergence slits, a secondary monochromator and a scintillation counter. The sample was prepared for analysis by packing the powder on to an aluminum sample holder. The specimen was rotated by 60.00 rpm and scanned by 4°/min at room temprature with Cu-ka radiation.

### Example 1

### 3-Hydroxymethyl)-N,N,2-trimethyl-8-[(5-methyl-3,4-dihydro-2H-chromen-4-yl)amino]imidazo[1,2a]pyridine-6-carboxamide

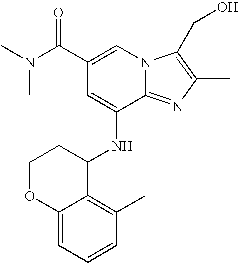

### STEP 1: 4-Chloro-5-methylchromane

A solution of thionyl chloride (81 mL, 1.1 mol) in diethyl ether (370 mL) was added to a mixture of 5-methylchroman-4-ol (61 g, 370 mmol, Tetrahedron Asym., 1997, 8, 3059.) and pyridine (1.4 mL) in diethyl ether (80 mL) and chloroform (200 mL) at 0° C. The reaction mixture was stirred at room temperature for 13 hours. After the mixture was evaporated in vacuo, the residue was poured into ice-water and extracted with ethyl acetate (500 mL×2). The combined extracts were washed with brine, dried over magnesium sulfate, and concentrated in vacuo to afford the title compound as yellow oil (68 g, quantitative yield).

¹H NMR (CDCl₃, 300 MHz) δ: 7.21-7.04 (m, 1H), 6.86-6.62 (m, 2H), 5.36-5.17 (m, 1H), 4.59-4.43 (m, 1H), 4.43-4.30 (m, 1H), 2.41 (s, 3H), 2.57-2.24 (m, 2H) ppm.

### STEP 2: Isopropyl 8-amino-2-methylimidazo[1,2-a]pyridine-6-carboxylate

To a solution of isopropyl 5,6-diaminonicotinate (65 g, 333 mmol) in cyclohexanone (500 mL) was added bromoacetone (51 g, 333 mmol) at room temperature. The reaction mixture was stirred at 95° C. for 2 hours. After the mixture was cooled to 0° C., the resulting precipitate was filtered and washed with n-hexane (500 mL) and diisopropylether (500 mL). The solids were dissolved in dichloromethane (1000 mL) and saturated sodium bicarbonate solution (800 mL). The organic layer was separated, dried over magnesium sulfate and concentrated in vacuo. The residue was purified by column chromatography on silica gel (dichloromethane/ethyl acetate=1/1 as eluent) to afford the title compound as brown syrup (43 g, 55%).

¹H NMR (CDCl₃, 270 MHz) δ: 8.30 (d, J=1.3 Hz, 1H), 7.33 (s, 1H), 6.84 (d, J=1.3 Hz, 1H), 5.35-5.15 (m, 1H), 4.60-4.39 (m, 2H), 2.45 (s, 3H), 1.37 (d, J=6.0 Hz, 6H) ppm.

MS (ESI) m/z: 234 (M+H)⁺. STEP 3: Isopropyl 2-methyl-8-[(5-methyl-3,4-dihydro-2H-chromen-4-yl)amino]imidazo[1,2a]pyridine-6-c arboxylate

To a mixture of isopropyl 8-amino-2-methylimidazo[1,2-a]pyridine-6-carboxylate (43 g, 183 mmol, STEP 2), sodium iodide (14 g, 91 mmol) and potassium carbonate (88 g, 640 mmol) in acetone (480 mL) was added a solution of 4-chloro-5-methylchromane (50 g, 274 mmol, STEP 1) in acetone (80 mL) at 45° C. and the mixture was stirred at 56° C. for 15 hours. After cooled to room temperature, the mixture was quenched with water (300 mL) and extracted with dichloromethane (500 mL×2). The combined extracts were dried over magnesium sulfate, and evaporated in vacua. The residue was washed with n-hexane (300 mL), 2-propanol I diisopropylether (20 mL/200 mL) and methanol (80 mL) to afford the title compound as a yellow solid (30 g, 43%).

¹H NMR (CDCl₃, 300 MHz) δ: 8.26 (s, 1H), 7.31 (s, 1H), 7.12 (t, J=8.1 Hz, 1H), 6.85-6.68 (m, 3H), 5.36-5.21 (m, 2H), 4.78-4.67 (m, 1H), 4.33-4.15 (m, 2H), 2.39 (s, 3H), 2.35-2.00 (m, 5H), 1.40 (d, J=5.9 Hz, 6H) ppm.

MS (ESI) m/z: 380 (M+H)⁺.

### STEP 4: 2-Methyl-8-](5-methyl-3,4-dihydro-2H-chromen-4-yl)amino]imidazo[1,2-a]pyridine-6-carboxylic acid

A mixture of isopropyl 2-methyl-8-[(5-methyl-3,4-dihydro-2H-chromen-4-yl)amino]imidazo[1,2-a]pyridine-6-carboxylate (8.6 g, 23 mmol, STEP 3) and 2M sodium hydroxide solution (34 mL) in methanol (15 mL) and tetrahydrofran (15 mL) was stirred at 60° C. for 0.5 hour. After cooled to room temperature, the mixture was neutralized with 2M hydrochloric acid (34 mL). The resulting precipitate was collected by filtration and dried to afford the title compound as a white solid (7.5 g, 98%).

¹H NMR (DMSO-d₆, 270 MHz) δ: 8.52 (s, 1H), 7.72 (s, 1H), 7.13 (t, J=7.9 Hz, 1H), 6.83-6.66 (m, 3H), 5.71-5.62 (m, 1H), 4.86-4.75 (m, 1H), 4.30-4.06 (m, 2H), 2.28 (s, 3H), 2.20-1.85 (m, 5H) ppm (−COOH was not observed)

MS (ESI) m/z: 338 (M+H)⁺, 336 (M−H)⁻.

### STEP 5: N,N,2-Trimethyl-8-[(5-methyl-3,4-dihydro-2H-chromen-4-yl)amino]imidazo[1,2-a]pyridine-6-carboxamide

To a stirred mixture of 2-methyl-8-[(5-methyl-3,4-dihydro-2H-chromen-4-yl)amino]imidazo[1,2-a]pyridine-6-carboxylic acid (7.5 g, 22 mmol, STEP 4), N-methylmethanamine hydrochloride (2.7 g, 33 mmol), 1-hydroxybenzotriazole hydrate (HOBt) (4.1 g, 27 mmol) and triethylamine (9.3 mL, 67 mmol) in dichloromethane (110 mL) was added 1-(3-dimethylaminopropyl)-3-ethylcarbodiimide hydrochloride (EDCl) (5.1 g, 27 mmol) at 0° C. and the reaction mixture was stirred at room temperature for 1 day. To the reaction mixture was added water and extracted with dichloromethane. The extract was washed with brine, dried over sodium sulfate, and evaporated in vacuco. The residue was purified by column chromatography on silica gel (dichloromethane/ethyl acetate=1/2 to 1/3 as eluent) to afford the title compound as a white solid (8.1 g, quantitative yield).

¹H NMR (CDCl₃, 300 MHz) δ: 7.63 (s, 1H), 7.27 (s, 1H), 7.12 (t, J=8.1 Hz, 1H), 6.75 (t, J=8.1 Hz, 2H), 6.26 (s, 1H), 5.36 (d, J=6.6 Hz, 1H), 4.69-4.61 (m, 1H), 4.31-4.17 (m, 2H), 3.13 (s, 6H), 2.38 (s, 3H), 2.32-2.15 (m, 4H), 2.12-1.95 (m, 1H) ppm.

MS (ESI) m/z: 365 (M+H)⁺, 363 (M−H)⁻.

### STEP 6: 3-(Hydroxymethyl)-N,N,2-trimethyl-8-](5-methyl-3,4-dihydro-2H-chromen-4-yl)amino]imidazo [1,2a]pyridine-6-carboxamide (example 1-1)

A mixture of N,N,2-trimethyl-8-[(5-methyl-3,4-dihydro-2H-chromen-4-yl)amino]imidazo[1,2-a]pyridine-6-carboxamide (8.1 g, 22 mmol, STEP 5), formaldehyde 37 wt. % in water (18 g, 222 mmol), acetic acid (3.2 mL, 56 mmol) and sodium acetate (4.6 g, 56 mmol) in acetonitrile (220 mL) was heated at 80° C. for 1.3 hours. After cooled to room temperature, saturated sodium bicarbonate solution (200 mL) was added to the reaction mixture and extracted with ethylacetate (200 mL×2). The combined extracts were washed with brine, dried over sodium sulfate, and evaporated in vacuo. The residue was purified by column chromatography on silica gel (dichloromethane/methanol=20/1 as eluent) to afford the title compound as a white solid (8.4 g, 95%).

¹H NMR (CDCl₃, 300 MHz) δ: 7.77 (s, 1H), 7.12 (t, J=8.1 Hz, 1H), 6.75 (t, J=8.1 Hz, 2H), 6.35 (s, 1H), 5.38 (d, J=6.6 Hz, 1H), 4.88 (s, 2H), 4.72-4.62 (m, 1H), 4.33-4.16 (m, 2H), 3.13 (s, 6H), 2.37 (s, 3H), 2.31-2.14 (m, 4H), 2.14-1.98 (m, 1H), 1.88-1.78 (m, 1H) ppm.

MS (ESI) m/z: 395 (M+H)⁺, 393 (M−H)⁻.

### STEP 7: (S)-(−)-3-(Hydroxymethyl)-N,N,2-trimethyl-8-[(5-methyl-3,4-dihydro-2H-chromen-4-yl)amino]imdazo[1,2-a]pyridine-6-carboxamide fraction-1)and

### (R)-(+)-3-(Hydroxymethyl)-N,N,2-trimethyl-8-[(5-methyl-3,4-dihydro-2H-chromen-4-yl)amino]imidazo[1,2a]pyridine-6-carboxamide (fraction-2)

The fraction-1 (2.46 g) and fraction-2 (2.39 g) were prepared from racemic 3-(hydroxymethyl)-N,N,2-trimethyl-8-[(5-methyl-3,4-dihydro-2H-chromen-4-yl)amino]imidazo[1,2-a]pyridine-6- carboxamide 5.9 g) by HPLC as follows.

### Isolation Condition

Column: CHIRALPAK® OD-H (20 mm I.D.×250 mm, DAICEL)

Mobile phase: n-Hexane/Ethanol/Diethylamine (85/15/0.1)

Flow rate: 18.9 mL/min

### (S)-(−)-3-(Hydroxymethyl)-N,N,2-trimethyl-8-[(5-methyl-3,4-dihydro-2H-chromen-4-yl)amino]imidazo[1,2-a]pyridine-6-carboxamide (fraction-1) (example 1-2)

NMR: spectrum data were identical with those of the racemate

optical rotation: [α]_{D}²²=−5.3° (C=1.03, Methanol)

retention time: 8 min
(R)-(−)-3-(Hydroxymethyl)-N,N,2-trimethyl-8-[(5-methyl-3,4-dihydro-2H-chromen-4-yl)amino]imidazo[1,2-a]pyridine-6-carboxamide (fraction-2) (example1-3)

NMR: spectrum data were identical with those of the racemate

optical rotation: [α]_{D}²¹=+6.0° (C=1.08, Methanol)

retention time: 14 min

### Example 2

### 8-(3,4-Dihydro-2H-chromen-4-ylamino)-3-(hydroxymethyl)-N,N,2-trimethylimidazo[1,2-a]pyridine-6-carboxamide

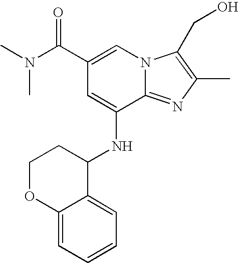

### STEP 1: Isopropyl 8-(3,4-dihydro-2H-chromen-4-ylamino)-2-methylimidazo[1,2-a]pyridine-6-carboxylate

The title compound was prepared in 93% yield (10.2 g, oil) from 4-chlorochromane (7.6 g, 45 mmol, Indian Journal of Chemistry, Section B, 1981, 20B(12), 1063.) and isopropyl 8-amino-2-methylimidazo[1,2-a]pyridine-6-carboxylate (7.0 g, 30 mmol, STEP 2 of Example 1) by the same manner in STEP 3 of Example 1.

¹H NMR (CDCl₃, 300 MHz) δ: 8.26 (s, 1H), 7.37-7.17 (m, 3H), 6.98-6.82 (m, 2H), 6.77 (s, 1H), 5.47-5.38 (m, 1H), 5.35-5.21 (m, 1H), 4.87-4.76 (m, 1H), 4.33-4.23 (m, 2H), 2.40 (s, 3H), 2.30-1.95 (m, 2H), 1.39 (d, J=5.9 Hz, 6H) ppm.

MS (ESI) m/z: 366 (M+H)⁺.

### STEP 2. Isopropyl 8-(3,4-dihydro-2H-chromen-4-ylamino)-3-(hydroxymethyl)-2-methylimidazo[1,2-a]pyri dine-6-carboxylate

The title compound was prepared in 63% yield (7.0 g, a white solid) from isopropyl 8-(3,4-dihydro-2H-chromen-4-ylamino)-2-methylimidazo[1,2-alpyridine-6-carboxylate (10.2 g, 27.9 mmol, STEP 1) by the same manner in STEP 6 of Example 1.

¹H NMR (CDCl₃, 300 MHz) δ: 8.37 (s, 1H), 7.40-7.14 (m, 2H), 6.95-6.81 (m, 3H), 5.44-5.37 (m, 1H), 5.36-5.22 (m, 1H), 4.97 (d, J=5.1 Hz, 2H), 4.88-4.79 (m, 1H), 4.33-4.24 (m, 2H), 2.42 (s, 3H), 2.30-2.20 (m, 2H), 1.40 (d, J=6.6 Hz, 6H) ppm. (—OH was not observed)

MS (ESI) m/z: 396 (M+H)⁺.

### STEP 3: 8-(3,4-Dihydro-2H-chromen-4-ylamino)-3-(hydroxymethyl)-2-methylimidazo[1,2-a]pyridine-6-car boxylic acid

The title compound was prepared in quantitative yield (4.8 g, a yellow solid) from isopropyl 8-(3,4-dihydro-2H-chromen-4-ylamino)-3-(hydroxymethyl)-2-methylimidazo[1,2-a]pyridine-6-carboxylate (5.1 g, 12.9 mmol, STEP 2) by the same manner in STEP 4 of Example 1.

¹H NMR (DMSO-d₆, 300 MHz) δ: 13.2-12.9 (m, 1H), 8.35 (s, 1H), 7.31-7.07 (m, 2H), 6.93-6.68 (m, 3H), 6.20-5.90 (m, 1H), 5.30-5.13 (m, 1H), 5.08-4.90 (m, 1H), 4.84-4.66 (m, 2H), 4.36-4.13 (m, 2 H), 2.32 (s, 3H), 2.24-2.01 (m, 2H) ppm.

MS (ESI) m/z: 354 (M+H)⁺, 352 (M−H)⁻.

### STEP 4: 8-(3,4-Dihydro-2H-chromen-4-ylamino)-3-(hydroxymethyl)-N,N,2-trimethylimidazo[1,2a]pyridine-6-carboxamide (example 2-1)

To a stirred mixture of 8-(3,4-dihydro-2H-chromen-4-ylamino)-3-(hydroxymethyl)-2-methylimidazo[1,2-a]pyridine-6-carboxylic acid (760 mg, STEP 3) and N-methylmethanamine hydrochloride (370 mg, 4.5 mmol) and triethylamine (0.84 mL, 6.0 mmol) in dimethylformamide (15 mL) was added O-benzotriazol-1-yl-N,N,N′,N′-tetramethyluronium hexafluorophosphate (HBTU) (1.1 g, 3.0 mmol) at 0° C. The reaction mixture was stirred at room temperature for 3 hours. To the reaction mixture was added water and the mixture was extracted with ethylacetate. The extract was washed with brine, dried over sodium sulfate, and evaporated in vacuo. The residue was purified by column chromatography on silica gel (methanol/dichloromethane=1/20 as eluent) to afford the title compound as a white solid (344 mg).

¹H NMR (CDCl₃, 300 MHz) δ: 7.75 (s, 1H), 7.34-7.16 (m, 2H), 6.94-6.82 (m, 2H), 6.30 (s, 1H), 5.52 (d, J=6.6 Hz, 1H), 4.93-4.82 (m, 2H), 4.81-4.72 (m, 1H), 4.33-4.22 (m, 2H), 3.10 (s, 6H), 2.46-2.10 (m, 5H) ppm. (—OH was not observed)

MS (ESI) m/z: 381 (M+H)⁺, 379 (M−H)⁻.

### STEP 5: (+)-8-(3,4-Dihydro-2H-chromen-4-ylamino)-3-(hydroxymethyl)-N,N,2-trimethylimidazo[1,2-a]pyridine-6-carboxamide (fraction-1) and

### (−)-8-(3,4-Dihydro-2H-chromen-4-ylamino)-3-(hydroxymethyl)-N,N,2-trimethylimidazo[1,2-a]pyridine-6-car boxamide (fraction-2)

The fraction-1 (132 mg) and fraction-2 (130 mg) were prepared from racemic 8-(3,4-dihydro-2H-chromen-4-ylamino)-3-(hydroxymethyl )-N,N,2-trimethylimidazo[1,2-a]pyridine-6-carbox amide (335 mg) by HPLC as follows.

### Isolation Condition

Column: CHIRALPAK® OD-H (20 mm I.D.×250 mm, DAICEL)

Mobile phase: n-Hexane/Ethanol/Diethylamine (85/15/0.1)

Flow rate: 18.9 mL/min

### (+)-8-(3,4-Dihydro-2H-chromen-4-ylamino)-3-(hydroxymethyl)-N,N,2-trimethylimidazo[1,2-a]pyridine-6-car boxamide (fraction-1) example 2-2)

NMR: spectrum data were identical with those of the racemate

optical rotation: [α]_{D}²¹=+12.3 (C=0.20, Methanol) retention time: 8 min

### (−)-8-(3,4-Dihydro-2H-chromen-4-ylamino)-3-hydroxymethyl)-N,N,2-trimethylimidazo[1,2-a]pyridine-6-car boxamide (fraction-2) (example 2-3)

NMR: spectrum data were identical with those of the racemate

optical rotation: [α]_{D}²¹=−10.0° (C=0.27, Methanol)

retention time: 13 min

### Example 3

### 8-[(5,7-Difluoro-3,4-dihydro-2H-chromen-4-yl)amino]-3-(hydroxymethyl)-N,N,2-trimethylimidazo[1,2-a]pyridine-6-carboxamide

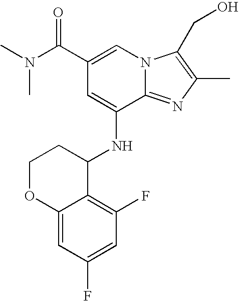

### STEP 1: 5,7-Difluorochroman-4-ol

To a stirred solution of 5,7-difluoro-2,3-dihydro-4H-chromen-4-one (2.0 g, 11 mmol, US 2005038032) in methanol (30 mL) was added sodium borohydride (0.49 g, 13 mmol) at 0° C. and the mixture was stirred at room temperature for 20 hours. After the mixture was evaporated in vacuo, the residue was treated with water (20 mL) and extracted with ethyl acetate (30 mL×2). The combined extracts were washed with brine, dried over magnesium sulfate, and concentrated in vacuo to afford the title compound as a white solid (2.0 g, 97%).

¹H NMR (CDCl₃, 270 MHz) δ: 6.50-6.33 (m, 2H), 5.07-4.95 (m,1H), 4.36-4.18 (m, 2H), 2.16-1.94 (m, 2H) ppm. (—OH was not observed)

### STEP 2: 4-Chloro-5,7-difluorochromane

The title compound was prepared in quantitative yield (2.1 g, yellow oil) from 5,7-difluorochroman-4-ol (2.0 g, 11 mmol, STEP 1) by the same manner in STEP 1 of Example 1.

¹H NMR (CDCl₃, 300 MHz) δ: 6.56-6.30 (m, 2H), 5.45-5.25 (m, 1H), 4.62-4.33 (m, 2H), 2.53-2.20 (m, 2H) ppm.

### STEP 3: Isopropyl 8-[(5,7-difluoro-3,4-dihydro-2H-chromen-4-yl)amino]-2-methylimidazo[1,2-a]pyridine-6-carboxylate

The title compound was prepared in 82% yield (2.8 g, a yellow solid) from isopropyl 8-amino-2-methylimidazo[1,2-a]pyridine-6-carboxylate (1.6 g, 7.0 mmol, STEP 2 of Example 1) 4-chloro-5,7-difluorochromane (2.1 g, 11 mmol, STEP 2) by the same manner in STEP 3 of Example 1.

¹H NMR (CDGl₃, 300 MHz) δ: 8.28 (s, 1H), 7.32 (s, 1H), 6.78 (s, 1H), 6.48-6.34 (m, 2H), 5.37-5.20 (m, 2H), 4.98-4.89 (m, 1H), 4.38-4.23 (m, 2H), 2.41 (s, 3H), 2.36-2.24 (m, 1H), 2.21-2.01 (m, 1H), 1.39 (d, J=6.6 Hz, 6H) ppm.

MS (ESI) m/z: 402 (M+H)⁺.

### STEP 4: 8-[5,7-Difluoro-3,4-dihydro-2H-chromen-4-yl)amino]-2-methylimidazo[1,2-a]pyridine-6-carboxylic acid

The title compound was prepared in 64% yield (1.5 g, a yellow solid) from isopropyl 8-[(5,7-difluoro-3,4-dihydro-2H-chromen-4-yl)amino]-2-methylimidazo[1,2-a]pyridine-6-carboxylate (2.8 g, 6.8 mmol, STEP 3) by the same manner in STEP 4 of Example 1.

¹H NMR (DMSO-d₆, 300 MHz) δ: 8.37 (s, 1H), 7.66 (s, 1H), 6.83-6.67 (m, 2H), 6.67-6.48 (m, 1H), 6.02 (d, J=7.3 Hz, 1H), 4.99-4.86 (m,1H), 4.37-4.15 (m, 2H), 2.27 (s, 3H), 2.17-1.83 (m, 2H) ppm. (—COOH was not observed)

MS (ESI) m/z: 360 (M+H)⁺.

### STEP 5: 8-[(5,7-Difluoro-3,4-dihydro-2H-chromen-4-yl)amino]-N,N,2-trimethylimidazo[1,2-a]pyridine-6-carboxyamide

The title compound was prepared in 92% yield (0.79 g, a white solid) from 8-[(5,7-difluoro-3,4-dihydro-2H-chromen-4-yl)amino]-2-methylimidazo]1,2-a]pyridine-6-carboxylic acid (0.80 g, 2.2 mmol, STEP 4) by the same manner in STEP 5 of Example 1.

¹H NMR (CDCl₁, 300 MHz) δ: 7.64 (s, 1H), 7.27 (s, 1H), 6.50-6.33 (m, 2H), 6.26 (s, 1H), 6.35 (d, J=5.8 Hz, 1H), 4.91-4.80 (m, 1H), 4.36-4.25 (m, 2H), 3.12 (s, 6H), 2.40 (s, 3H), 2.34-2.20 (m, 1H), 2.08-1.91 (m, 1H) ppm.

MS (ESI) m/z: 387 (M+H)⁺.

### STEP 6: 8-[(5,7-Difluoro-3,4-dihydro-2H-chromen-4-yl)amino]-3-(hydroxymethyl)-N,N,2-trimethylimidazo [1,2-a]pyridine-6-carboxamide (example 3-1)

The title compound was prepared in 94% yield (0.79 g, a white solid) from 8-[(5,7-difluoro-3,4-dihydro-3,4-dihydro-2H-chromen-4-yl)amino]-N,N,2-trimethylimidazo[1,2-a]pyridine-6-carboxamide (0.79 g, 2.0 mmol, STEP 5) by the same manner in STEP 6 of Example 1.

¹H NMR (CDCl₃, 270 MHz) δ: 7.76 (s, 1H), 6.52-6.25 (m, 3H), 5.40 (d, J=5.9 Hz, 1H), 4.97-4.76 (m, 3H), 4.41-4.18 (m, 2H), 3.12 (s, 6H), 2.34 (s, 3H), 2.32-2.12 (m, 2H), 2.11-1.91 (m, 1H) ppm.

MS (ESI) m/z: 417 (M+H)⁺.

### STEP 7: (R)-(+)-8-[(5,7-Difluoro-3,4-dihydro-2H-chromen-4-yl)amino]-3-(hydroxymethyl)-N,N,2-trimethylimidazo]1,2-a]pyridine-6-carboxamide (fraction-1) and

(S)-(−)-8-[(5,7-Difluoro-3,4-dihydro-2H-chromen-4-yl)amino]-3-hydroxymethyl)-N,N,2-trimethylimidazo[1,2-a]pyrdine-6-carboxamide (fraction-2)

The fraction-1 (0.25 g) and fraction-2 (0.26 g) were prepared from racemic 8-[(5,7-difluoro-3,4-dihydro-2H-chromen-4-yl)amino]-3-(hydroxymethyl)-N,N,2-trimethyimidazo[1,2-a]pyridine-6-carboxamide (0.78 g) by HPLC as follows.

### Isolation Condition

Column: CHIRALPAK® AD-H (20 mm I.D.×250 mm, DAICEL)

Mobile phase: n-Hexane/2-Propanol I Diethylamine ( 90/10/0.1)

flow rate: 18.9 mL/min

### (R)-(+)-8-[(5,7-Difluoro-3,4-dihydro-2H-chromen-4-yl)amino]-3-(hydroxymethyl)-N,N,2-trimethylimidazo[1, 2-a]pyridine-6-carboxamide fraction-1) (example 3-2)

NMR: spectrum data were identical with those of the racemate

optical rotation [α]_{D}²⁴=+48.7 (c=1.01, Methanol)

retention time: 13 min

### (S)-(−)-8-[(5,7-Difluoro-3,4-dihydro-2H-chromen-4-yl)amino]-3-(hydroxymethl)-N,N,2-trimethylimidazo[1,2-a]pyridine-6-carboxamide (fraction-2) (example 3-3)

NMR: spectrum data were identical with those of the racemate

optical rotation: [α]_{D}²⁴=31 49.9 (c=1.01, Methanol)

retention time: 18 min

mp: 186° C.

PXRD pattern angle (2-Theta°): 10.6, 13.0, 14.4, 16.7, 19.7, 22.6, 26.5

### Example 4

### 8-[(5-Fluoro-3,4-dihydro-2H-chromen-4yl)amino]-3-hydroxymethyl)-N,N,2-trimethylimidazo[1,2-a]pyridine-6-carboxamide

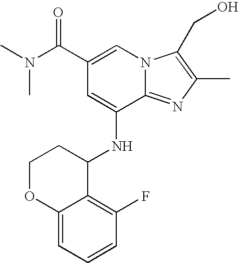

### STEP 1: 5-Fluorochroman-4-ol

The title compound was prepared as black oil in quantitative yield from 5-fluoro-2,3-dihydro-4H-chromen-4-one (GB 2355264) by the same manner in STEP 1 of Example 3.

¹H NMR (CDCl₃, 300 MHz) δ: 7.25-7.11 (m, 1H), 6.75-6.60 (m, 2H), 5.13-5.02 (m, 1H), 4.40-4.18 (m, 2H), 2.25-1.95 (m, 3H) ppm.

### STEP 2: 4-Chloro-5-fluorochromane

The title compound was prepared in quantitative yield (15 g, orange oil) from 5-fluorochroman-4-ol (13 g, 77 mmol, STEP 1) by the same manner in STEP 1 of Example 1.

¹H NMR (CDCl₃, 270 MHz) δ: 7.24-7.10 (m, 1H), 6.71-6.56 (m, 2H), 5.43-5.33 (m, 1H), 4.58-4.32 (m, 2H), 2.50-2.19 (m, 2H) ppm.

### STEP 3:

### Isopropyl 8-[(5-fluoro-3,4-dihydro-2H-chromen-4-yl)amino]-2-methylimidazo[1,2-a]pyridine-6-carboxylate

The title compound was prepared in 61% yield (12 g, a yellow solid) from 4-chloro-5-fluorochromane (14 g, 77 mmol, STEP 2 of Example 4) and isopropyl 8-amino-2-methylimidazo[1,2-alpyridine-6-carboxylate (2.2 g, 7.1 mmol, STEP 2 of Example 1) by the same manner in STEP 3 of Example 1.

¹H NMR(CDCl₃, 270 MHz) δ: 8.27(s, 1H) 7.31 (s, 1H), 7.24-7.10(m, 1H), 6.80(s, 1H), 6.74-6.57 (m, 2H), 5.40-5.21 (m, 2H), 5.04-4.93 (m,1H), 4.36-4.25 (m, 2H), 2.40 (s, 3H), 2.36-2.23 (m, 1H), 2.19-1.97 (m, 1H), 1.39 (d, J=5.9 Hz, 6H) ppm.

MS (ESI) m/z: 384 (M+H)⁺.

### STEP 4: 8-[5-Fluoro-3,4-dihydro-2H-chromen-4-yl)amino]-2-methylimidazo[1,2-a]pyridine-6-carboxylic acid

The title compound was prepared in 98% yield (9.5 g, a white solid) from isopropyl 8-[(5-fluoro-3,4-dihydro-2H-chromen-4-yl)amino]-2-methylimidazo[1,2-alpyridine-6-carboxylate (11 g, 28 mmol, STEP 3) by the same manner in STEP 4 of Example 1.

¹H NMR (DMSO-d₆, 300 MHz) δ: 8.51 (s, 1H), 7.72 (s, 1H), 7.32-7.16 (m, 1H), 6.78-6.64 (m, 3H), 6.12 (d, J=7.3 Hz, 1H), 5.06-4.94 (m, 1H), 4.35-4,15 (m, 2H), 2.29 (s, 3H), 2.16-1.93 (m, 2H) ppm. (—COOH was not observed)

### STEP 5; 8-[(5-Fluoro-3,4-dihydro-2H-chromen-4-yl)amino]-N,N,2-trimethylimidazo[1,2-a]pyridine-6-carboxamide

The title compound was prepared in 99% yield (0.67 g, a white solid) from 8-[(5-fluoro-3,4-dihydro-2H-chromen-4-yl)amino]-2-methylimidazo[1,2-a]pyridine-6-carboxylic acid (0.64 g, 1.9 mmol STEP 4) by the same manner in STEP 5 of Example 1.

¹H NMR (CDCl₃, 270 MHz) δ: 7.63 (s,1H), 7.33-7.23 (m, 1H), 7.24-7.10 (m,1H), 6.76-6.55 (m, 2H), 6.27 (s, 1H), 5.43 (d, J=5.8 Hz, 1H), 4.97-4.84 (m, 1H), 4.36-4.23 (m, 2H), 3.12 (s, 6H), 2.39 (s, 3H), 2.32-2.22 (m, 1H), 2.11-1.93 (m, 1H) ppm.

MS (ESI) m/z: 369 (M+H)⁺.

### STEP 6: 8-[(5-Fluoro-3,4-dihydro-2H-chromen-4-yl)amino]-N,N,2-trimethylimidazo[1,2-a]pyridine-6-carboxamide (fraction-1) and fraction-2)

The fraction-1 (0.25 g) and fraction-2 (0.25 g) were prepared from racemic 8-[(5-fluoro-3,4-dihydro-2H-chromen-4-yl)amino]-N,N,2-trimethylimidazo[1,2-a]pyridine-6-carboxamide (0.66 g) by HPLC as follows.

### Isolation Condition

Column: CHIRALPAK® OD-H (20 mm I.D.×250 mm, DAICEL)

Mobile phase: n-Hexane/EtOH/Diethylamine (80/20/0.1)

flow rate: 20 mL/min

### 8-[(5-Fluoro-3,4-dihydro-2H-chromen-4-yl)amino]-N,N,2-trimethylimidazo[1,2-a]pyridine-6-carboxamide (fraction-1)

NMR: spectrum data were identical with those of the racemate

retention time: 7 min

MS (ESI) m/z: 369 (M+H)⁺.

### 8-[(5-Fluoro-3,4-dihydro-2H-chromen-4-yl)amino]-N,N,2-trimethylimidazo[1,2-a]pyridine-6-carboxamide (fraction-2)

NMR; spectrum data were identical with those of the racemate

retention time: 11 min

MS (ESI) m/z: 369 (M+H)⁺.

### STEP 7: (−)-8-[(5-Fluoro-3,4-dihydro-2H-chromen-4-yl)amino]-3-(hydroxymethyl)-N,N,2-trimethylimidazo [1,2a]pyridine-6-carboxamide example 4-2)

The title compound was prepared in 93% yield (0.13 g, a white solid) from 8-[(5-fluoro-3,4-dihydro-2H-chromen-4-yl)amino]-N,N,2-trimethylimidazof1,2-a]pyridine-6-carboxamide (0.13 g, 0.35 mmol, fraction-1 of STEP 6) by the same manner in STEP 6 of Example 1.

¹H NMR (CDCl₃, 300 MHz) δ: 7.78 (s, 1H), 7.25-7.12 (m, 1H), 6.73-6.55 (m, 2H), 6.36 (s, 1H), 5.42 (d, J=5.8 Hz, 1H), 4.97-4.82 (m, 3H), 4.36-4.20 (m, 2H), 3.13 (s, 6H), 2.38 (s, 3H), 2.32-2.20 (m, 1H), 2.12-1.92 (m, 1H), 1.80-1.65 (m, 1H) ppm.

MS (ESI) m/z: 399 (M+H)⁺.

optical rotation: [α]_{D}²³=−49.7° (c=1.01, Methanol)

### STEP 8; (+)-8-[(5-Fluoro-3,4-dihydro-2H-chromen-4yl)-amino]-3-(hydroxymethyl)-N,N,2-trimethylimidazo [1,2-a]pyridine-6-carboxamide (example 4-3)

The title compound was prepared in 94% yield (0.13 g, a white solid) from 8-[(5-fluoro-3,4-dihydro-2H-chromen-4-yl)amino]-N,N,2-trimethylimidazo[1,2-a]pyridine-6-carboxamide (0.13 g, 0.35 mmol, fraction-2 of STEP 6) by the same manner in STEP 6 of Example 1.

¹H NMR (CDCl₃, 300 MHz) δ: 7.78 (s, 1H), 7.24-7.10 (m, 1H), 6.73-6.56 (m, 2H), 6.36 (s, 1H), 5.42 (d, J=5.8 Hz, 1 H), 4.97-4.83 (m, 3H), 4.36-4.19 (m, 2H), 3.13 (s, 6H), 2.39 (s, 3H), 2.34-2.21 (m, 1H), 2.12-1.92 (m, 1H), 1.69-1.53 (m, 1H) ppm.

MS (ESI) m/z: 399 (M+H)⁺.

optical rotation: [α]_{D}²⁴=+54.3° (c=1.01, Methanol)

### Example 5

### (S)-3-(Hydroxymethyl)-N,N,2-trimethyl-8-[(5-methyl-3,4-dihydro-2H-chromen-4-yl)amino]imidazo[1, 2-a]pyridine-6-carboxamide

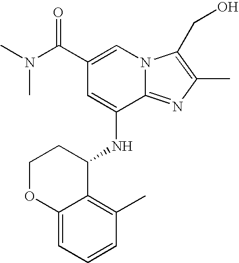

### STEP 1; Methyl 3-(2-chloro-5-methylphenoxy)acrylate

A solution of 2-chloro-5-methylphenol (10.0 g, 70.1 mmol) and methyl propiolate (5.95 mL, 71.5 mmol) in acetonitrile (30 mL) was added to a stirred solution of TBAF in THF (1.0 M commercial solution, 14 mL, 14 mmol) at room temperature over a period of 1 hour. After complete addition of the solution, stirring was continued for 1 hour. The reaction mixture was diluted with toluene (50 mL) and washed twice with water (50 mL+25 mL). Separated organic layer was concentrated under reduced pressure to afford the title compound as brown oil (17.2 g, >99%, 6 : 4 mixture of cis- and trans- isomers with ca. 10 wt % of toluene), which was used in the next step without further purification.

¹H NMR (CDCl₃, 300 MHz,) δ: 7.71 (d, J=12.5 Hz, 0.4H), 7.30 (m, 1H), 6.98-6.93 (m, 2H), 6.74 (d, J=7.3 Hz, 0.6H), 5.47 (d, J=12.5 Hz, 0.4H), 5.20 (d, J=7.3 Hz, 0.6H), 2.77 (s, 1.8H), 3.73 (s, 1.8H), 3.73 (s, 1.3H), 2.34-2.33 (two singlets, 3H) ppm.

### STEP 2: Methyl 3-(2-chloro-5-methylphenoxy)propanoate

A mixture of methyl 3-(2-chloro-5-methylphenoxy)acrylate (1.00 g, 4.41 mmol, STEP 1), sodium bromide (10 mg, 0.097 mmol) and 10% palladium on carbon (50 mg) in methanol (5 mL) was stirred overnight under H₂ (1 atm) at room temperature. The reaction mixture was filtered through Celite® pad, and catalyst was rinsed with toluene (10 mL). The combined filtrate was washed with water (5 mL) and concentrated under reduced pressure to afford the title compound (963 mg, 95%) as orange oil, which was used in the next step without further purification.

¹H NMR (CDCl₃, 300 MHz) δ: 7.21 (d, J=8.1 Hz, 1H), 6.78 (br.s, 1H), 6.73 (br.d, J=8.8 Hz, 1H), 4.30 (t, J=6.6 Hz, 2H), 3.74 (s, 3H), 2.86 (t, J=6.6 Hz, 2H), 2.32 (s, 3H) ppm.

### STEP 3: 8-Chloro-5-methyl-2,3-dihydro-4H-chromen-4-one

A mixture of methyl 3-(2-chloro-5-methylphenoxy)propanoate (430 mg, 1.88 mmol, STEP 2) and trifluoromethanesulfonic acid (0.86 mL, 2 mL/g of substrate) was stirred at 80° C. for 40 minutes. After cooling to room temperature, the reaction mixture was diluted with water, and product was extracted with toluene. Organic layer was washed successively with aqueous solution of K₂CO₃ and water, and concentrated under reduced pressure to afford the title compound (355 mg, 96%) as a pale brown solid, which was used in the next step without further purification.

¹H NMR (CDCl₃, 300 MHz) δ: 7.41 (d, J=8.1 Hz, 1H), 6.76 (d, J=8.1 Hz, 1H), 4.61 (t, J=6.6 Hz, 2H), 2.85 (t, J=6.6 Hz, 2H) 2.61 (s, 3H) ppm.

### STEP 4: (4S)-8-Chloro-5-methyl-N-[(1S)-1-phenylethyl]chroman-4-amine 4-amine 4-methylbenzenesulfonate

To a solution of 8-chloro-5-methyl-2,3-dihydro-4H-chromen-4-one (1.97 g, 10 mmol, STEP 3) in tetrahydrofuran (4 mL) were added (S)-1-phenylethylamine (1.64 mL, 13 mmol) and titanium(IV) isopropoxide(4.44 mL, 15 mmol) at 22° C. The yellow solution was stirred at 22° C. for 18 hours. After completion of the reaction (checked by ¹H-NMR), the mixture was diluted with methanol (20 mL) and cooled to approximately −30° C. To this solution was added a 2.0 M solution of sodium borohydride in triglyme (2.5 mL, 5 mmol) over 30 minutes (internal temperature was kept between −20 and −25 ° C.) under nitrogen. The reaction mixture was stirred at −20 ° C. for 30 min, and then 10% w/v aqueous solution of sodium citrate (35 mL) was added. This yellow mixture was stirred vigorously at 22° C. for 5 minutes, and then ethyl acetate (60 mL) was added. The resulting mixture was stirred at 22° C. for 15 hours and two layers were separated. The organic layer was washed with 5% w/v aqueous solution of sodium chloride (20 mL) and concentrated. Crude product (69.8% de by HPLC) was dissolved in methanol (65 mL), and the solution was warmed to 70° C. (external temperature). To this yellow solution was added dropwise an aqueous solution of 4-methylbenzenesulfonic acid monohydrate (2.47 g, 13 mmol in 15 mL of water) over 10 minutes. Additional water (45 mL) was added, and the mixture was cooled slowly to 22° C. and stirred overnight (12 hours) at 22° C. After filtration, the white solid was washed with ethyl acetate (20 mL), and then dried in vacuum oven at 50° C. for 2 hours to afford the title compound (2.82 g, 59%, 99.3% de) as a white solid.

¹H NMR (DMSO-d₆, 300 MHz) δ: 8.98 (br.s, 1H), 8.71 (br.s, 1H), 7.65 (d, J=6.6 Hz, 2H), 7.49-7.46 (m, 5H), 7.39 (d, J=8.1 Hz, 1H), 7.12 (d, J=7.3 Hz, 2H), 6.87 (d, J=8.1 Hz, 1H), 4.72-4.68 (m, 2H), 4.42-4.32 (m, 2H), 2.40 (s, 3H), 2.29 (s, 3H), 2.13-2.00 (m, 2H), 1.69 (d, J=5.8 Hz, 3H) ppm.

### Analytical Conditions (HPLC)

Column: Xterra MS C18 3.5 μm (2.1 mm I.D.×150 mm, Waters).

Temperature: 40° C.

Detection: UV (230 nm)

Mobile phase: CH₃CN (A), 10 mM CH₃COONH₄ (B). Gradient table is given below.

| Time (min) | % A | % B | Flow rate (mL/min) |
|---|---|---|---|
| 0.0 | 20 | 80 | 0.3 |
| 25.0 | 95 | 5 | 0.3 |
| 30.0 | 95 | 5 | 0.3 |

Retention Time

Fraction 1: 21.8 min (undesired diastereomer)

Fraction 2: 22.6 min (desired diastereomer)

### STEP 5: (4S)-5-Methylchroman-4-amine hydrochloride

To a suspension of (4S)-8-chloro-5-methyl-N-[(1 S)-1-phenylethyl]chroman-4-amine 4-methybenzenesulfonate (2.37 g, 5.0 mmol, STEP 4) in ethyl acetate (19 mL) was added 1 M aqueous solution of sodium hydroxide (10 mL) at 22° C. The suspension was stirred vigorously at 22° C. for 10 minutes. Two layers were separated. The organic layer was washed with water (5 mL) and concentrated to afford a free amine as colorless oil. The free amine was dissolved in methanol (20 mL) and the solution was hydrogenolyzed in the presence of 10% palladium on carbon (31 mg) at 50° C. for 3 hours under hydrogen (1 atm). After the reaction mixture was cooled down to 22° C., the catalyst was filtered off through a Celite® pad and washed with methanol. The filtrate was concentrated to afford the title compound (1.00 g, 100%, 99.4% ee) as a white solid.

¹H NMR (DMSO-d₆, 300 MHz) δ: 8.52 (brs, 3H), 7.17 (t, J=8.0 Hz, 1H), 6.79 (d, J=7.0 Hz, 1H), 6.70 (d, J=14.7 Hz, 1H), 2.05-2.20 (m, 8.0 Hz, 1H), 4.55 (s, 1H), 4.32 (d, J=10.3 Hz, 2H), 2.39 (s, 3H), 2.30 (d, J=14.7 Hz, 1H), 2.05-2.20 (m, 1H) ppm.

### Analytical Conditions (HPLC)

Column: CHIRALPAK® AD-H

Temperature: 40° C.

Detection: UV (230 nm)

Mobile phase: n-Hexane/Ethanol/Diethylamine ( 90/10/0.1)

Flow rate: 1.0 mL/min

Retention time
Fraction 1: 8.0 min (R-isomer)
Fraction 2: 9.6 min (S-isomer)

### STEP 6: Isopropyl 2-methyl-8-{[(4S)-5-methyl-3,4-dihydro-2H-chromen-4-yl]amino}imidazol[1,2-a]pyridine-6-carboxylate

### STEP 6-1: Isopropyl 6-amino -5-bromonicotinate

To a 500 mL 3-necked flask containing a suspension of isopropyl 6-aminonicotinate (14.9 g, 82.5 mmol) in cyclopentylmethylether (CPME) (150 mL) was added NBS in seven portions (2.93 g×7, 116 mmol as a whole) with intervals of 10 minutes at 22° C. After 15 minutes of stirring, reaction was quenched with 3% aqueous solution of sodium thiosulfate (Na₂S₂0₃) (150 mL) and 5% aqueous solution of NaHCO₃ (150 mL). To this mixture was added toluene (300 mL), and the mixture was stirred for 10 minutes. Separated organic layer was concentrated under reduced pressure and the solvent was chased with 2-propanol (90 mL×2) and partially concentrated to 75 mL. The mixture was stirred at room temperature for 15 hours then at 0° C. for 5 hours. Resulting solid was filtered, and was washed twice with cold 2-propanol (30 mL) to afford the title compound (16.4 g, 63.3 mmol, 77%) as a yellow brown solid.

¹H NMR(CDCl₃, 300 MHz) δ: 8.66(s, 1H), 8.24 (s, 1H), 5.40 (br.s, 2H), 5.22 (sep, J=6.6 Hz, 1H), 1.35 (d, J=6.6 Hz, 6H) ppm.

### STEP 6-2: Isopropyl 8-bromo-2-methylimidazo[1,2-a]pyridine-6-carboxylate

A mixture of isopropyl 6-amino-5-bromonicotinate (15.0 g, 57.9 mmol, STEP 6-1), chloroacetone (14.0 mL, 174 mmol), and propionitrile (150 mL) was stirred at 100 ° C. After 71 hours of stirring, chloroacetone (4.7 mL, 58 mmol) was added, and stirring was continued for 24 hours at the same temperature. Then another portion of chloroacetone (4.7 mL, 58 mmol) and propionitrile (60 mL) were added. After stirring for 9 hours, the reaction mixture was cooled to room temperature, and then quenched with 0.5 M NaOH solution (116 mL) and water (34 mL). To this mixture was added toluene (150 mL), and the mixture was stirred for 15 minutes. Separated organic layer was concentrated under reduced pressure and the solvent was chased with mixed solvent (heptane : ethyl acetate =1 : 1, 50 mL×2). The residue was diluted with 1: 1 mixture of heptane and ethyl acetate (300 mL) and silica gel (30 g) was added. After stirring for 10 minutes, the mixture was filtered and washed with 1: 1 mixture of heptane and ethyl acetate (150 mL×2). Filtrate was concentrated under reduced pressure. The solvent was chased with 2-propanol (150 mL×2) and partially concentrated to approximately 20 mL. Heptane (70 mL) was added, and the mixture was stirred at room temperature for 1 hour and then at 0° C. for 3 hours. Resulting solid was filtered and washed twice with 19:1 mixture of heptane and 2-propanol (30 mL) to afford the title compound (8.3 g, 28 mmol, 48%) as a pale milky brown solid.

¹H NMR (CDCl₃, 300 MHz) δ: 8.78 (d, J=1.4 Hz, 1H) 7.96 (s, 1H), 7.50 (s, J=8 Hz, 1H), 5.28 (sep, J=5.8 Hz, 1H), 2.52 (s, 3H), 1.39 (d, J=5.8 Hz, 6H) ppm.

### STEP 6-3: Isopropyl 2-methyl-8-{[(4S)-5-methyl-3,4-dihydro-2H-chromen-4-yl]amino}imidazo[1,2-a]pyridine-6-carboxlate

The two-necked round-bottomed flask (20 mL) equipped with a reflux condenser was charged with Pd₂(dba)₃ (3.7 mg, 0.004 mmol) and BINAP (5.6 mg, 0.009 mmol), and purged with nitrogen. Toluene (1 mL) was added, and the mixture was stirred at 22° C. for 5 minutes, resulting in a heterogeneous purple solution. (4S)-5-Methylchroman-4-amine hydrochloride (80 mg, 0.4 mmol, STEP 5), sodium tert-butoxide (85 mg, 0.88 mmol) and toluene (1 mL) were added and the mixture was stirred at 60° C. for 5 minutes. isopropyl 8-bromo-2-methylimidazo[1,2-a]pyridine-6-carboxylate (119 mg, 0.4 mmol, STEP 6-2) and toluene (1 mL) were added and then the mixture was stirred at 80° C. for 5 hours. The reaction mixture was allowed to cool to 22° C. and then diluted with diisopropyl ether (3 mL). The resulting suspension was filtered through Celite® pad, and filtrate was concentrated under reduced pressure. Crude product was purified by silica-gel column chromatography (heptane: ethyl acetate=4:1) to afford the title compound (118 mg, 78%) as a light pink powder.

¹H NMR (CDCl₃, 300 MHz) δ: 8.26 (s, 1H), 7.32 (s, 1H), 7.12 (t, J=8.1 Hz, 1H), 6.78-6.72 (m, 3H), 5.32-5.24 (m, 2H), 4.73 (br., 1H), 4.27-4.19 (m, 2H), 2.39 (s, 3H), 2.29 (d, J=15.0 Hz, 1H), 2.22 (s, 3H), 2.14-2.09 (m, 1H), 1 40 (d, J=5.8 Hz, 6H) ppm.

### STEP 7: 2-Methyl-8-{[(4S)-5-methyl-3,4-dihydro-2H-chromen-4-yl]amino}imidazo[1,2-a]pyridine-6-carbox ylic acid

The title compound is prepared from isopropyl 2-methyl-8-{[(4S)-5-methyl-3,4-dihydro-2H-chromen-4-yl]amino}imidazo[1,2-a]pyridine-6-carboxylate (STEP 6-3) by the same manner in STEP 4 of Example 1.

### STEP 8: N,N,2-Trimethyl-8-{[(4S)-5-methyl-3,4-dihydro-2H-chromen-4-yl]amino}imidazo[1,2-a]pyridine-6-carboxamide

The title compound is prepared from 2-methyl-8-{[(4S)-5-methyl-3,4-dihydro-2H-chromen-4-yl]amino}imidazo[1,2-a]pyridine-6-carboxylic acid (STEP 7) by the same manner in STEP 5 of Example 1.

### STEP 9: 3-(Hydroxymethyl)-N,N,2-trimethyl-8-{[(4S)-5-methyl-3,4-dihydro-2H-chromen-4-yl]amino}imidazo[1,2-a]pyridine-6-carboxamide

The title compound is prepared from N,N,2-trimethyl-8-{[(4S)-5-methyl-3,4-dihydro- 2H-chromen-4-yl]amino}imidazo[1,2-a]pyridine-6-carboxamide (STEP 8) by the same manner in STEP 6 of Example 1.

### Example 6

### [2-Methyl-8-[(5-methyl-3,4-dihydro-2H-chromen-4-yl)amino]-6-(morpholin-4-ylcarbonyl)imidazo[1,2-a]pyridin-3-yl]methanol

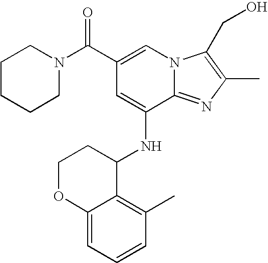

### STEP 1:

### 2-Methyl-N-(5-methyl-3,4-dihydro-2H-chromen-4-yl)-6-(morpholin-4-ylcarbonyl)imidazo[1,2-a]pyridin-8-amine

To a stirred mixture of 2-methyl-8-[(5-methyl-3,4-dihydro-2H-chromen-4-yl )amino]imidazo[1,2-a]pyridine-6-carboxylic acid (0.60 g, 1.8 mmol, STEP 4 of Example 1) and morpholine (0.31 g, 3.6 mmol) in dichloromethane (8.0 mL) were added 1-hydroxybenzotriazole hydrate (HOBt) (0.36 g, 2.7 mmol) and 1-(3-dimethylaminopropyl)-3-ethylcarbodiimide hydrochloride (EDCI) (0.51 g, 2.7 mmol) at 0° C. and the reaction mixture was stirred at room temperature for 20 hours. The reaction mixture was quenched with saturated sodium hydrogencarbonate solution and extracted with dichloromethane (30 mL×2). The combined extracts were washed with brine, dried over sodium sulfate, and evaporated in vacuo. The residue was purified by column chromatography on silica gel (hexane/ethyl acetate=1/2 as eluent) to afford the title compound as a white solid (0.73 g, quantitative yield).

¹H NMR (CDCl₃, 300 MHz) δ: 7.64 (s, 1H), 7.28 (s, 1H), 7.18-7.07 (m, 1H), 6.80-6.69 (m, 2H), 6.21 (s, 1H), 5.41 (d, J=5.8 Hz, 1H), 4.70-4.60 (m, 1H), 4.34-4.17 (m, 2H), 3.81-3.61 (m, 8H), 2.38 (s, 3H), 2.22 (s, 3H), 2.31-1.95 (m, 2H) ppm.

MS (ESI) m/z: 407 (M+H)⁺.

### STEP 2:

### 2-Methyl-N-(5-methyl-3,4-dihydro-2H-chromen-4-yl)-6-morpholin-4-ylcarbonyl)imidazo[1,2-a]pyridin-8-amine (fraction-1 and fraction-2)

The fraction-1 (0.27 g) and fraction-2 (0.28 g) were prepared from racemic 2-methyl-N-(5-methyl-3,4-dihydro-2H-chromen-4-yl)-6-( morpholin-4-ylcarbonyl )imidazo[1,2-a]pyridin-8-am ine (0.72 g) by HPLC as follows.

### Isolation Condition

Column; CHIRALPAK® OJ-H (20 mm I.D.×250 mm, DAICEL)

Mobile phase: n-Hexane Ethanol/Diethylamine (65/35/0.1)

Flow rate: 20 mL/min

### 2-Methyl-N-(5-methyl-3,4-dihydro-2H-chromen-4-yl)-6-(morpholin-4-ylcarbonyl)imidazo[1,2-a]pyridin-8-am ine (fraction-1)

NMR; spectrum data were identical with those of the racemate

MS (ESI) m/z: 407 (M+H)⁺.

retention time: 8 min

### 2-Methyl-N-(5-methyl-3,4-dihydro-2H-chromen-4-yl)-6-(morpholin-4-ylcarbonyl)imidazo[1,2-a]pyridin-8-am ine (fraction-2)

NMR: spectrum data were identical with those of the racemate

MS (ESI) m/z: 407 (M+H)⁺.

retention time: 14 min

### STEP 3:

### (−)-[2-Methyl-8-(5-methyl-3,4-dihydro-2H-chromen-4-yl)amino]-6-(morpholin-4-ylcarbonyl)imidazo[1,2-a]p yridin-3-yl]methanol (example 6-2)

A mixture of 2-methyl-N-(5-methyl-3,4-dihydro-2H-chromen-4-yl)-6-(morpholin-4-ylcarbonyl)imidazo [1,2-a]pyridin-8-amine (0.22 g, 0.55 mmol, fraction-1 of STEP 2), formaldehyde 37 wt. % in water (0.45 g, 5.5 mmol), acetic acid (0.78 mL, 1.4mmol) and sodium acetate (0.11 g, 1.4 mmol) in acetonitrile (5 mL) was heated at 70° C. for 3 hours. After cooled to room temperature, the reaction mixture was quenched with 1 M sodium hydroxide solution and extracted with ethylacetate (20 mL×2). The combined extracts were washed with brine, dried over sodium sulfate, and evaporated in vacuo. The residue was purified by column chromatography on silica gel (dichloromethane I methanol=15/1 as eluent) and NH gel (ethylacetate as eluent) to afford the title compound as a white solid (0.18 g, 76%).

¹H NMR (CDCl₃, 300 MHz) δ: 7.76 (s, 1H), 7.13 (t, J=8.1 Hz, 1H), 6.81-6.69 (m, 2H), 6.30 (s, 1H), 5.46 (d, J=6.6 Hz, 1H), 4.91-4.78 (m, 2H), 4.70-4.60 (m, 1H), 4.33-4.17 (m, 2H), 3.87-3.60 (m, 8H), 2.49-2.38 (m, 1H), 2.33 (s, 3H), 2.21 (s, 3H), 2.29-2.15 (m, 1H), 2.15-1.97 (m, 1H) ppm.

MS (ESI) m/z: 437 (M+H)⁺.

optical rotation: [α]_{D}²³=−12.0° (c=1.01, Methanol)

### STEP 4.

### (+)-[2-Methyl-8-[(5-methyl-3,4-dihydro-2H-chromen-4-yl)amino]-6-(morpholin-4-ylcarbonyl)imidazo[1,2-a]pyridin-3-yl]methanol (example 6-3)

The title compound was prepared in 73% yield (0.18 g, a white solid) from 2-methyl-N-(5-methyl-3,4-dihydro-2H-chromen-4-yl)-6-(morpholin-4-ylcarbonyl)imidazo[1,2-a]pyridin-8-amine (0.23 g, 0.56 mmol, fraction-2 of STEP 2) by the same manner in STEP 3 of Example 6.

¹H NMR(CDCl₃, 300 MHz) δ: 7.76(s, 1H), 7.13(t, J=8.1 Hz, 1H), 6.81-6.69(m, 2H), 6.30 (s, 1H), 5.46 (d, J=6.6 Hz, 1H), 4.91-4.78 (m, 2H), 4.70-4.60 (m, 1H), 4.33-4.17 (m, 2H), 3.87-3.60 (m, 8H), 2.81-2.64 (m, 1H), 2.33 (s, 3H), 2.21 (s, 3H), 2.29-2.15 (m, 1H), 2.15-1.97 (m, 1H) ppm.

MS (ESI) m/z: 437 (M+H)⁺.

optical rotation: [α]_{D}²⁴=+11.8° (c=1.01, Methanol)

### Example 7

### [8-(3,4Dihydro-2H-chromen-4-ylamino)-2-methyl-6-(morpholin-4-ylcarbonyl)imidazo[1,2-a]pyridin-3-yl]methanol

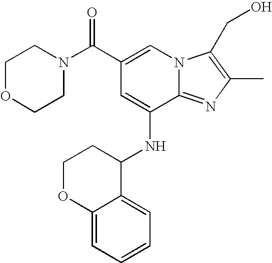

### STEP 1: [8-(3,4-Dihydro-2H-chromen-4-ylamino)_-2-methyl-6-(morpholin-4-ylcarbonyl)imidazo[1,2-a]pyridi n-3-yl]methanol (example 7-1)

To a stirred mixture of 8-(3,4-dihydro-2H-chromen-4-ylamino)-3-(hydroxymethyl)-2-methylimidazo[1,2-a]pyridine-6-carboxylic acid (2.4 g, 6.9 mmol, STEP 3 of Example 2), morpholine (1.8 g, 21 mmol) and triethylamine (1.44 mL, 10 mmol) in dimethylformamide (70 mL) was added O-benzotriazol-1-yl-N,N,N′,N′-tetramethyluronium hexafluorophosphate (HBTU) (3.9 g, 10 mmol) at 0° C. The reaction mixture was stirred at room temperature for 3 hours. To the reaction mixture was added water, and the mixture was extracted with ethyl acetate. The extract was washed with brine, dried over sodium sulfate, and evaporated in vacuo. The residue was purified by column chromatography on silica gel (ethyl acetate/methanol=20/1 as eluent) to afford the title compound as a white solid (1.6 g, 53%).

¹H NMR (CDCl₃, 300 MHz) δ: 7.78 (s, 1H), 7.33-7.16 (m, 2H), 6.94-6.82 (m, 2H), 6.25 (s, 1H), 5.57-5.50 (m, 1H), 4.94-4.86 (m, 2H), 480-4.70 (m, 1H) 4.32-4.23 (m, 2H), 3.80-3.60 (m, 8H), 2.40 (s, 3H), 2.30-2.15 (m, 2H) ppm. (—OH was not observed)

MS (ESI) m/z: 423 (M+H)⁺, 421 (M−H)⁻.

### STEP 2:

### (−)-[8-(3,4-Dihydro-2H-chromen-4-ylamino)-2-methyl-6-(morpholin-4-ylcarbonyl)imidazo[1,2-a]pyridin-3-yl]methanol (fraction-1) and

### (+)-[8-(3,4-Dihydro-2H-chromen-4-ylamino)-2-methyl-6-(morpholin-4-ylcarbonyl)imidazo[1,2-a]pyridin-3-yl]methanol (fraction-2)

The fraction-1 (570 mg) and fraction-2 (570 mg) were prepared from racemic [8-(3,4-dihydro-2H-chromen4-ylamino)-2-methyl-6-(morpholin-4- ylcarbonyl)imidazo[1,2-a]pyridin-3-yl]met hanol (1.4 g) by HPLC as follows.

### Isolation Condition

Column: CHIRALPAK® AD-H (20 mm I.D.×250 mm, DAICEL)

Mobile phase: n-Hexane/2-Propanol/Diethylamine (85/15/0.1)

Flow rate: 18.9 mL/min

### (−)-[f8-(3,4-Dihydro-2H-chromen-4-ylamino)-2-methyl-6-(morpholin-4-ylcarbonyl)imidazo[1,2-a]pyridin-3-yl]methanol (fraction-1) (example 7-2)

NMR: spectrum data were identical with those of the racemate

optical rotation: [α]_{D}²⁴=−3.21° (C=1.00, Methanol)

retention time: 16 min

### (+)-[8-(3,4-Dihydro-2H-chromen-4-ylamino)-2-methyl-6-(morpholin-4-ylcarbonyl)imidazo[1,2-a]pyridin-3-yl]methanol (fraction-2) (example 7-3)

NMR: spectrum data were identical with those of the racemate

optical rotation: [α]_{D}²⁵=+4.21° (C=0.91, Methanol)

retention time: 19 min

### Example 8

### [8-[(5,7-Difluoro-3,4-dihydro-2H-chromen-4-yl)amino]-2-methyl-6-(morpholin-4-ylcarbonyl)imidazo[1,2-a]pyridin-3-yl]methanol

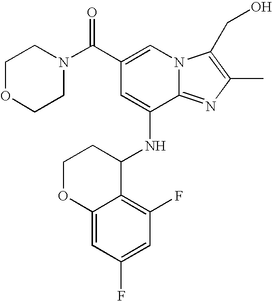

### STEP 1:

### N-(5,7-Difluoro-3,4-dihydro-2H-chromen-4-yl)-2-methyl-6-(morpholin-4-ylcarbonyl)imidazo[1,2-a]pyridin-8-amine

The title compound was prepared in 75% yield (4.49 g, a white solid) from 8-[(5,7-difluoro-3,4-dihydro-2H-chromen-4-yl)amino]-2-methylimidazo[1,2-a]pyridine-6-carboxylic acid (5.00 g, 13.9 mmol, STEP 4 of Example 3) by the same manner in STEP 1 of Example 6.

¹H NMR (CDCl₃, 300 MHz) δ: 7.64 (d, J=1.3 Hz, 1H), 7.27 (s, 1H), 6.47-6.36 (m, 2H), 6.22 (s, 1H), 5.40 (d, J=6.6 Hz, 1H), 4.95 (m, 1H), 4.35-4.23 (m, 2H), 3.71 (m, 8H), 2.39 (s, 3H), 2.30-2.22 (m, 1H), 2.09-1.95 (m, 1H) ppm.

MS (ESI) m/z: 429 (M+H)⁺.

### STEP 2:

### [8-[5,7-Difluoro-3,4-dihydro-2H-chromen-4-yl)amino]-2-methyl-6-(morpholin-4-ylcarbonyl)imidazo[1,2-a]p yrin-3-yl]methanol (example 8-1)

The title compound was prepared in 97% yield (4.68 g, a white solid) from N-(5,7-Difluoro-3,4-dihydro-2H-chromen-4-yl)-2-methyl-6-(morpholin-4-ylcarbonyl)imidazo[1,2-a]pyridin-8-amine (4.49 g, 10.5 mmol, STEP 1) by the same manner in STEP 3 of Example 6.

¹H NMR (CDCl₃, 300 MHz) δ: 7.76 (s, 1H) 6.45-6.35 (m, 2H), 6.30 (s, 1H), 5.48 (d, J=6.6 Hz, 1H), 4.86 (s, 3H), 4.92-4.85 (m, 1H), 4.38-4.22 (m, 2H), 3.71 (m, 8H), 2.33 (s, 3H), 2.33 (m, 1H), 2.10-1.95 (m, 1H) ppm.

MS (ESI) m/z: 459 (M+H)⁺.

### STEP 3:

### (+)-[8-[(5,7-difluoro-3,4-dihydro-2H-chromen-4-yl)amino]-2-methyl-6-(morpholin-4-ylcarbonyl)imidazo[1,2-a]pyridin-3-yl]methanol (fraction-1) and

### (−)-[8-[(5,7-Difluoro-3,4-dihydro-2H-chromen-4-yl)amino]-2-methyl-6-(morpholin-4-ylcarbonyl)imidazo[1,2-a]pyridin-3-ylmethanol (fraction-2)

The fraction-1 (0.49 9) and fraction-2 (0.48 g) were prepared from racemic [8-[(5,7-difluoro-3,4-dihydro-2H-chromen-4-yl)amino]-2-methyl-6-(morpholin-4-ylcarbonyl)imidazo[1,2-a]py ridin-3-yl]methanol (1.50 g) by HPLC as follows,

### Isolation Condition

Column: CHIRALPAK® AD-H (20 mm I.D.×250 mm, DAICEL)

Mobile phase: n-Hexane/Ethanol/Diethylamine (85/15/10.1)

flow rate: 18.9 mL/min

### (+)-[8-[(5,7-Difluoro-3,4-dihydro-2H-chromen-4-yl)amino]-2-methyl-6-(morpholin-4-ylcarbonyl)imidazo[1,2-a]pyridin-3-yl]methanol (fraction-1) (example 8-2)

NMR: spectrum data were identical with those of the racemate

optical rotation: [α]_{D}²³=+54.2° (c=1.20, Methanol)

retention time: 11 min

### (−)-[8-[5,7-Difluoro-3,4-dihydro-2H-chromen-4-yl)amino]-2-methyl-6-(morpholin-4-ylcarbonyl)imidazo[1,2-a]pyridin-3-ylmethanol (fraction-2) (example 8-3)

NMR: spectrum data were identical with those of the racemate

optical rotation: [α]_{D}²⁴=−51.2 (c=1.34, Methanol)

retention time: 18 min

### Example 9

### [8-[5-Fluoro-3,4-dihydro-2H-chromen-4-yl)amino]-2-methyl-6-(morpholin-4-ylcarbonyl)imidazo[1,2-a]pyridin-3-yl]methanol

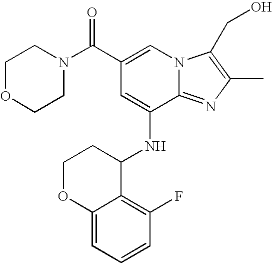

### STEP 1:

### N-(5-Fluoro-3,4-dihydro-2H-chromen-4-yl)-2-methyl-6-(morpholin-4-ylcarbonyl)imidazo[1,2-a]pyridin-8-amine

The title compound was prepared in 96% yield (4.5 g, a pale brown solid) from 8-[(5-fluoro-3,4-dihydro-2H-chromen-4-yl)amino]-2-methylimidazo[1,2-a]pyridine-6-carboxylic acid (3.8 g, 11 mmol, STEP 4 of Example 4) by the same manner in STEP 1 of Example 6.

¹H NMR (CDCl₃, 300 MHz) δ: 7.64 (s, 1H), 7.35-7.10 (m, 2H), 6.78-6.57 (m, 2H), 6.29 (s, 1H), 5.80-5.68 (m, 1H), 5.00-4.85 (m, 1H), 4.40-4.27 (m, 2H), 3.88-3.61 (m, 8H), 2.39 (s, 3H), 2.33-1.84 (m, 2H) ppm.

MS (ESI) m/z: 411 (M+H)⁺.

### STEP 2:

### [8-[(5-Fluoro-3,4-dihydro-2H-chromen-4-yl)amino]-2-methyl-6-(morpholin-4-ylcarbonyl)imidazo[1,2-a]pyridin-3-yl]methanol (example 9-1)

The title compound was prepared in 93% yield (4.4 g, a white solid) from N-(5-Fluoro-3,4-dihydro-2H-chromen-4-yl)-2-methyl-6-(morpholin-4-ylcarbonyl)imidazo[1,2-a]pyridin-8-am ine (4.5 g, 11 mmol, STEP 1) by the same manner in STEP 3 of Example 6.

¹H NMR (CDCl₃, 270 MHz) δ: 7.77 (s, 1H), 7.24-7.12 (m, 1H), 6.72-6.57 (m, 2H), 6.32 (s, 1H), 5.50-5.45 (m, 1H) 4.94-4.84 (m, 3H), 4.37-4.22 (m, 2H), 3.81-3.61 (m, 8H), 2.35 (s, 3H), 2.30-2.20 (m, 1H), 2.12-1.98 (m, 1H) ppm. (—OH was not observed)

MS (ESI) m/z: 441 (M+H)⁺.

### STEP 3:

### (+)-[8-[(5-Fluoro-3,4-dihydro-2H-chromen-4-yl)amino]-2-methyl-6-(morpholin-4-ylcarbonyl)imidazol[1,2-a]p yrin-3-ylmethanol (fraction-1) and

### (−)-[8-[(5-Fluoro-3,4-dihydro-2H-chromen-4-yl)amino]-2-methyl-6-(morpholin-4-ylcarbonyl)imidazo[1,2-a]p yridin-3-yl]methanol (fraction-2)

The fraction-1 (0.59 g) and fraction-2 (0.61 g) were prepared from racemic [8-[(5-fluoro-3,4-dihydro-2H-chromen-4-yl)amino]-2-methyl-6-(morpholin-4-ylcarbonyl)imidazo[1,2-a]pyridi n-3-yl]methanol (1.5 g) by HPLC as follows.

### Isolation Condition

Column: CHIRALPAK® AD-H (20 mm I.D.×250 mm, DAICEL)

Mobile phase: n-Hexane/2-Propanol/Diethylamine (80/20/0.1)

flow rate: 20 mL/min

### (+)-[8-[5-Fluoro-3,4-dihydro-2H-chromen-4-yl)amino]-2-methyl-6-(morpholin-4-ylcarbonyl)imidazo[1,2-a]p yridin-3-yl]methanol (fraction-1) (example 9-2)

NMR: spectrum data were identical with those of the racemate

optical rotation: [α]_{D}²⁴=+51.7° (c=1.04, Methanol)

retention time: 7 min

### (−)-[-[(5-Fluoro-3,4-dihydro-2H-chromen-4-ylamino]-2-methyl-6-(morpholin-4-ylcarbonyl)imidazo[1,2-a]p yridin-3-ylmethanol (fraction-2) (example 9-3)

NMR: spectrum data were identical with those of the racemate

optical rotation: [α]_{D}²⁴=−53.1° (c=1.04, Methanol)

retention time: 10 min

All publications, including but not limited to, issued patents, patent applications, and journal articles, cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications could be made without departing from the spirit of the invention.

1-10. (canceled)

11. A compound of the formula (I): 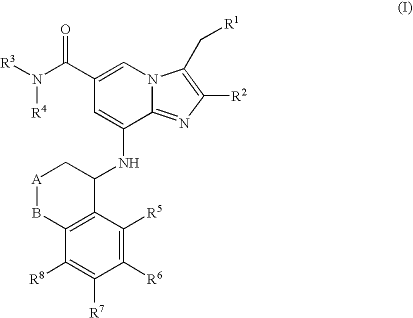
or a pharmaceutically acceptable salt thereof, wherein:
-A-B— represents —O—CH₂— or —CH₂—O—;
R¹ represents a hydroxy group or a moiety convertible into a hydroxy group in vivo;
R² represents a C₁-C₆ alkyl group;
R³ and R⁴ independently represent a C₁-C₆ alkyl group or a C₃-C₇ cycloalkyl group, said C₁-C₆ alkyl group and said C₃-C₇cycloalkyl group being unsubstituted or substituted with 1 to 3 substituents independently selected from the group consisting of a halogen atom, a hydroxy group, a C₁-C₆ alkoxy group and a C₃-C₇ cycloalkyl group; or R³ and R⁴ taken together with the nitrogen atom to which they are attached form a 4 to 7 membered heterocyclic group being unsubstituted or substituted with 1 to 3 substituents selected from the group consisting of a hydroxy group, a C₁-C₃ alkyl group, a C₁-C₆ alkoxy group and a hydroxy-C₁-C₆ alkyl group; and
R⁵, R⁶, R⁷ and R⁸ independently represent a hydrogen atom, a halogen atom or a C₁-C₆ alkyl group.

12. The compound or the pharmaceutically acceptable salt, as claimed in claim 11, wherein:
R¹ is a hydroxy group, C₁-C₆ alkoxy group or C₁-C₆ alkyl-carbonyl-oxy group; and
R³ and R⁴ are independently a C₁-C₆ alkyl group or a C₃-C7 cycloalkyl group, said C₁-C₆ alkyl group and said C₃-C₇cycloalkyl group being unsubstituted or substituted with 1 to 3 substituents independently selected from the group consisting of a halogen atom, a hydroxy group, a C₁-C₆ alkoxy group and a C₃-C₇ cycloalkyl group, or R³ and R⁴ taken together with the nitrogen atom to which they are attached form an azetidinyl group, a pyrrolidinyl group, a morpholino group or a homomorpholino group, said azetidinyl group, said pyrrolidinyl group, said morpholino group and said homomorpholino group being unsubstituted or substituted with 1 to 3 substituents selected from the group consisting of a hydroxy group, a C₁-C₆ alkyl group, a C₁-C₆ alkoxy group and a hydroxy-C₁-C₆ alkyl group.

13. The compound or the pharmaceutically acceptable salt, as claimed in claim 11, wherein:
-A-B— is —CH₂—O—;
R¹ is a hydroxy group;
R², R³ and R⁴ are independently a C₁-C₆ alkyl group;
R⁵ and R⁷ are independently a hydrogen atom, a halogen atom or a C₁-C₆ alkyl group; and
R⁶ and R⁸ are independently a hydrogen atom or a halogen atom.

14. A compound selected from:
(S)-(−)-3-(Hydroxymethyl )-N,N,2-trimethyl-8-[(5-methyl-3,4-dihydro-2H-chromen-4-yl)amino]imidazo[1,2-a]pyridine-6-carboxamide;
(+)-8-(3,4-Dihydro-2H-chromen-4-ylamino)-3-(hydroxymethyl)-N,N,2-trimethylimidazo[1,2-a]pyridine-6-carboxamide;
(S)-(−)-8-[(5,7-Difluoro-3,4-dihydro-2H-chromen4-yl)amino]-3-(hydroxymethyl)-N,N,2-trimethylimidazo[1,2-a]pyridine-6-carboxamide; and
(−)-8-[(5-Fluoro-3,4-dihydro-2H-chromen-4-yl)amino]-3-(hydroxymethyl)-N,N,2-trimethylimidazo[1,2-a]pyridine-6-carboxamide;
or a pharmaceutically acceptable salt thereof.

15. A pharmaceutical composition comprising the compound or the pharmaceutically acceptable salt thereof of claim 11, and a pharmaceutically acceptable carrier.

16. A pharmaceutical composition comprising the compound or the pharmaceutically acceptable salt thereof of claim 14, and a pharmaceutically acceptable carrier.

17. The pharmaceutical composition of claim 16 further comprising other pharmacologically active agent(s).

18. A method of treating a condition mediated by acid pump inhibitory activity in a mammalian subject including a human, which comprises administering to a mammal in need of such treatment a therapeutically effective amount of the compound or the pharmaceutically acceptable salt thereof, as claimed in claim 14.

19. A method of treating gastrointestinal disease, gastroesophageal disease, gastroesophageal reflux disease (GERD), laryngopharyngeal reflux disease, peptic ulcer, gastric ulcer, duodenal ulcer, NSAID-induced ulcers, gastritis, infection of Helicobacter pylori, dyspepsia, functional dyspepsia, Zollinger-Ellison syndrome, non-erosive reflux disease (NERD), visceral pain, cancer, heartburn, nausea, esophagitis, dysphagia, hypersalivation, airway disorders or asthma, which comprises administering to a mammal in need of such treatment a therapeutically effective amount of the compound or the pharmaceutically acceptable salt thereof, as claimed in claim 14.

20. A method of treating gastroesophageal reflux disease (GERD) comprising administering to a mammal in need of such treatment a therapeutically effective amount of the compound or the pharmaceutically acceptable salt thereof, as claimed in claim 14.
